(19) United States
(12) Patent Application Publication
(10) Pub. No.: US 2026/0227395 A1
(43) Pub. Date: Aug. 6, 2026

(54) BIOSENSOR MATERIALS

(71) Applicant: The Regents of the University of California, Oakland, CA (US)

(72) Inventors: Gang SUN, Davis, CA (US); Bofeng PAN, Davis, CA (US); Nitin NITIN, Davis, CA (US); Ahmed EL-MOGHAZY, Davis, CA (US)

(21) Appl. No.: 19/160,258

(22) PCT Filed: Feb. 20, 2024
(86) PCT No.: PCT/US2024/016525

§ 371 Date: Aug. 27, 2025

Related U.S. Application Data
(60) Provisional application No. 63/448,961, filed on Feb. 28, 2023.

(51) Int. Cl. G01N 33/545; G01N 33/543; G01N 33/569

(52) U.S. Cl. CPC G01N 33/545; G01N 33/54353; G01N 33/56916; G01N 2333/245; G01N 2469/10; G01N 2470/04; G01N 2470/10

(57) ABSTRACT

The disclosure provides a three-dimensional (3-D) macroporous melamine foam membrane comprising an amine reactive group. The membrane foam can be used to perform ELISA assays for the detection of an analyte. Diffusion of the analyte through the different membrane layers is fast and homogeneous in all directions and suitable for detection of large volume of samples in low concentration of targets and multiple targets simultaneously in one integrated system with minimal interference from targets.

### CROSS-REFERENCE TO RELATED APPLICATIONS

This application claims priority to U.S. Provisional Application No. 63/448,961, filed Feb. 28, 2023, the contents of which are hereby incorporated by reference in its entirety for all purposes.

### BACKGROUND

The Enzyme-linked Immunosorbent Assay (ELISA) is an immunological assay commonly applied to detect a variety of target analytes, such as antibodies, pesticides, antibiotics, proteins, and the like. Nowadays, conventional ELISA sensors have been widely employed in detection of hazardous chemicals in a broad range of areas, including food quality, environmental safety, biomedical diagnosis, and chemical controls. However, certain limitations of ELISA sensing materials exist, such as being expensive, time-consuming, lack of scalability and flexibility for on-site and simultaneous examinations of multi-target chemicals in very low concentrations, and dependence on specialized instrumentation. Besides, many conventional biosensors suffer from low sensitivity and inaccuracy due to the limitations of small and flat surfaces of the sensing media.

As an alternative, paper-based ELISA (p-ELISA) is a suitable fibrous and microporous platform with the advantage of high surface areas of fibers, low cost, ease of use, and low naked-eye distinguishable level. However, large biomolecules, such as antibodies and proteins, have difficulty to diffuse into and penetrate through the microporous media, which have heterogenous structures in vertical directions than the planar ones, resulting in less than expected amounts of biomolecules incorporated onto surfaces of fibers inside the media. Such a structural feature consequently lowers sensitivity, especially the p-ELISA sensors made of nitrocellulose, filter paper, and even nanofibrous membrane. The actual available active sites in these media are increased in rather limited amount, much lower than expectation, because of the structural limitations of the media, which also causes inhomogeneous colorimetric results in most p-ELISA signals.

As an example, a study on diffusion of large molecules through nanofibrous and porous membranes revealed that effective pores of nanofibrous membranes that are responsible for transport of biomolecules could be 1000 times smaller than the measured pore sizes of the membranes. The amount of antibody molecules loaded into the inside of the nanofibrous membranes could be significantly lower than those on the outside layers of the membranes, due to such a structural feature of nanofibrous membrane media. Thus, we envisioned that an ideal media for p-ELISA sensors should be three-dimensional homogenous and open, allowing large biomolecules to travel freely inside in all directions.

Macroporous aerogels produced from hydrogels or framework materials are considered an alternative sensing media, due to the existence of large pores. However, a strong binding potential between the hydrogel (aerogel) and aqueous solution would cause large amounts of non-specific adsorption of molecules, leading to a high false-positive rate and reduced sensitivity and accuracy in diagnostic applications. Most of the aerogels are not structurally homogeneous in three dimensions, and the macroporous structures retain a solid wall structure inside, blocking large molecules from moving freely from certain directions.

Reticulated melamine foams (MF) has some desired properties such as an open cell structure, hydrophilicity, high porosity, high nitrogen content, low flammability, high elasticity, and excellent mechanical properties, which are useful in an ELISA substrate. So far, MFs have been chemically modified for a wide range of applications in water treatments, such as oil/water separation, water disinfection, adsorption, strain/stress sensing, catalysis, and so on.

What is needed in the art are new materials which have high porosity and excellent mechanical properties that can be used for ELISA substrate sensors. The present disclosure satisfies this need and offers other advantages as well.

### BRIEF SUMMARY

The present disclosure provides a modified macroporous framework of melamine foams (MF) membranes for use in assays with pore sizes of about 60 μm to about 150 μm, which can accelerate the mass transfer of large biomolecules within the media, resulting in high homogeneity and fast response speed to target molecules. Advantageously, the high content of secondary amine structures in MF enables varied chemical modifications of the material for convenient covalent immobilization of biomolecules for immunoassay interactions. The hydrophilic nature of MF retains water molecules in its structure, ensuring adequate contact between analytes and surfaces of the material and rapid removal of unbounded molecules. The open cell interconnected framework structure with up to about 99% porosity provides high surface area, easy access to, and increased loading of biomolecules.

As such, in one embodiment, the present disclosure provides a three-dimensional (3-D) macroporous melamine foam membrane, which has been modified using an amine reactive coupling reagent (cross-linking agent) to generate a secondary amine reactive group.

In certain aspects, the amine reactive coupling reagent is a member selected from the group of an activated ester, an activated ester, a maleimide or a pyridyldithiol. An activated ester can be installed a cross-linking agent such as N, N′-disuccinimidyl carbonate (DSC).

In another embodiment, the disclosure provides a sandwich ELISA method for determining the presence of an analyte in solution, the method comprising:
contacting a macroporous melamine foam membrane comprising an immobilized capture antibody with a sample containing an analyte, wherein the capture antibody is specific for an epitope of the analyte (e.g., antigen) to form a captured analyte;
contacting the captured analyte with a detection antibody to form a sandwiched moiety; and detecting an output signal from the sandwiched moiety.

In another embodiment, the present disclosure provides a direct ELISA method for determining the presence of an analyte in a sample solution, the method comprising:
contacting a macroporous melamine foam membrane comprising an immobilized analyte with a capture antibody, wherein the capture antibody is specific for an epitope of the analyte in the sample to form a captured analyte; and
detecting an output signal from the captured analyte.

In yet another embodiment, the disclosure provides a competitive ELISA method for determining the amount an analyte in sample, the method comprising:
contacting a macroporous melamine foam membrane comprising an immobilized capture antibody with a sample containing the analyte and a conjugated analyte, wherein the analyte competes with the conjugated analyte in the sample for the immobilized capture antibody to form a captured analyte; and
detecting an output signal from the captured analyte.

In yet another embodiment, the disclosure provides a competitive ELISA method for determining the amount a foodborne pathogen in a sample, the method comprising:
contacting a macroporous melamine foam membrane comprising an immobilized capture antibody with a sample containing the foodborne pathogen and a conjugated foodborne pathogen, wherein the conjugated foodborne pathogen competes with the foodborne pathogen in the sample for the capture antibody to form a captured analyte; and
detecting an output signal from the captured foodborne pathogen.

These and other aspects, object and embodiments will become more apparent when read with the detailed description and the figures that follow.

### BRIEF DESCRIPTION OF THE DRAWINGS

FIG. 1A illustrates a scheme of a side-by-side chamber of the disclosure; FIG. 1B illustrates the correlation between time and concentration of IgG (150 KDa) and FIG. 1C illustrates the correlation between time and concentration of FITC-Dextran (40 KDa) inside receptor chamber with NF, NP, and different thicknesses of MF membranes (1 mm, 2 mm, and 3 mm). (NF=nanofibrous membrane, thickness=0.21 mm: NP=nitrocellulose paper, thickness=0.18 mm).

FIG. 2A illustrates a schematic of the preparation process of NHS@MF and protein immobilization on NHS@MF. FIG. 2B is an optical view of commercial MF and standardized membranes with a thickness of 1 mm and diameter of 5 mm. FIG. 2C illustrates a reaction of MF with DSC and proteins. FIG. 2D illustrates FTIR results of MF at different steps: pristine MF, NHS@MF, and protein immobilized NHS@MF. FIG. 2E illustrates water contact angles of MF and NHS@MF. FIG. 2F is a SEM image of MF, FIG. 2G is a SEM image of NHS@MF, and FIG. 2H is a SEM image of protein immobilized NHS@MF. Chemical structure of melamine foam and SEM-EDS results in FIG. 2I for carbon, FIG. 2J for nitrogen and FIG. 2K for oxygen. FIG. 2L shows a direct ELISA, FIG. 2M shows a sandwich ELISA, and FIG. 2N shows a competitive ELISA. The figure key is shown in FIG. 2O.

FIG. 3A illustrates protein immobilization distribution visualized by a laser scanning confocal microscope. FIG. 3B illustrates loaded NHS amount on NHS@MF and NHS@NF after the modification of DSC (5%). FIG. 3C illustrates immobilized antibody amounts on NHS@MF from 5 mg/L, 1 mg/L, and 0.5 mg/L of 100 μL antibody solution. FIG. 3D illustrates optical image and colorimetric signals generated from the interaction between immobilized HRP and TMB substrate on NHS@MF and pristine MF. FIG. 3E-F illustrates calibration curves for FIG. 3E shows HIgG, FIG. 3F shows FITC-dextran, FIG. 3G NHS, and FIG. 3H shows an antibody conjugated with Alexa 647.

FIG. 4A illustrates sensitivity of the assays. Optical images and the calibration curve of membranes in the detection of SARS-CoV-2 spike protein RBD using direct ELISA approach, and FIG. 4B illustrates sensitivity with a Sandwich ELISA and FIG. 4C illustrates sensitivity using an optical image and calibration curve of membranes treated by varied concentrations of CAP using a competitive ELISA approach.

FIG. 5A illustrates the effect of the sample volume in direct ELISA, FIG. 5B illustrates sandwich ELISA, and FIG. 5C illustrates competitive ELISA.

FIG. 6A illustrates the mechanism of simultaneous multiple on-site targets detection. FIG. 6B illustrates photographic demonstrated the fast-flow device driven by a syringe pump. FIG. 6C illustrates optical image and ARGB values of membranes treated by the mixture of varied concentrations of CAP and CPS using a competitive ELISA approach.

FIG. 7 illustrates the immobilized antibody amounts on NHS@MF, NHS@NF, and nitrocellulose paper (NP) from 1 mg/L of 100 μL of antibody solution.

FIG. 8A illustrates images of the NHS@MF membranes with different treatments after the addition of TMB substrate: 100 μL SP-RBD-His (2 mg/L): 200 μL BSA (3%); 100 μL SP-RBD-His (2 mg/L), and then 200 μL BSA (3%); 200 μL BSA (3%) and then 100 μL Ab-HIS-HRP (1 mg/L): 100 μL Ab-HIS-HRP (1 mg/L); 100 μL SP-RBD-His (2 mg/L), 200 μL BSA (3%), and then 100 μL Ab-HIS-HRP (1 mg/L). FIG. 8B illustrates a bar diagram for the ARGB observed from the images.

FIG. 9A illustrates optical images and the intensity of colorimetric signals of 1%, 3%. 5% BSA treated membranes and 1%, 3%. 5% skim milk treated membranes. FIG. 9B illustrates optical images of a sensing test of NHS@MF after the material was exposed to different concentrations of CAP. (without image crop).

FIG. 10A illustrates a calibration curve for E. coli O157:H7 at the wavelength of 600 nm; FIG. 10B is a photograph demonstrating the liquid filtering test using syringes and vials. FIG. 10C illustrates a chemical structure of melamine foam. FIG. 10D illustrates a reaction of MF with DSC and proteins and FIG. TOE illustrates FTIR results of MF and NHS@MF.

FIG. 11A illustrates a scheme of the used side-by-side chamber; FIG. 11B illustrates the correlation between diffusion time and concentration of E. coli O157:H7 inside receptor chamber with NF, NP, and different thicknesses of MF membranes. FIG. 11C illustrates a vertical flow test of E. coli O157:H7 solution (at 10⁴ CFU/mL concentration) through various martials: NF, NP and MF with different thicknesses, with each material positioned at the base of a syringe. FIG. 11D illustrates the unspecific adsorption of E. coli onto the tested materials after buffer wash. (NF thickness=0.21 mm; NP thickness=0.18 mm). Data are presented as mean±SD, with n=3 independent experiments. *LOD=Limit of detection

FIG. 12A is a schematic illustration of the procedure for foam-based sandwich ELISA (f-ELISA) with immobilizing antibodies and capturing bacteria; FIG. 12B shows adding HRP-labeled secondary antibody and enzymatic substrate TMB to generate color signals and obtaining images using a smartphone.

FIG. 13A illustrates SEM images of Pristine MF, FIG. 13B illustrates SEM images NHS@MF after incubation with E. coli O157:H7 solution (10⁵ CFU/mL): FIG. 13C illustrates SEM images Ab@NHS@MF incubated with E. coli O157:H7 solution (10⁵ CFU/mL); FIG. 13D illustrates SEM images (d) Ab@NHS@MF after incubation with E. coli O157:H7 solution (10³ CFU/mL). Fluorescent microscopic images of (FIG. 13E) NHS@MF and (FIG. 13F) Ab@NHS@MF, after incubation with E. coli O157:H7 solution at a concentration of 10⁷ CFU/mL. FIG. 13G shows data presented as mean±SD, with n=3 independent experiments. ***P<0.001 (two-tailed Student's t-test).

FIG. 14A illustrates optical images and the calibration curve of membranes in the detection of E. coli O157:H7. FIG. 14B illustrate a linear equation for the colorimetric assay was fitted to be y=0.0749x+26.499 (R²=0.989) between 10 and 1000 CFU/mL. Data are presented as mean z SD, with n=3 independent experiments.

FIG. 15A illustrates a calibration curves generated using varying sample volumes for the detection of E. coli O157:H7. FIG. 15B illustrates influence of sample volume on the colorimetric signal intensities in the f-ELISA method. Data are presented as mean±SD, with n=3 independent experiments.

FIG. 16 illustrates optical images and the colorimetric signals before and after 1 h enrichment of E. coli O157:H7 at various concentrations. Data are presented as mean±SD, with n=3 independent experiments. **P<0.01 (two-tailed Student's t-test).

FIG. 17 illustrates selectivity of f-ELISA toward E. coli O157:H7 detection in comparison with other bacteria strains, including Pseudomonas fluorescens. Listeria innocua, Listeria monocytogenes, Salmonella enterica and E. coli BL21. The concentration of each bacteria strain used in this experiment is 10⁵ CFU/mL. Data are presented as mean±SD, with n=3 independent experiments. ***P<0.001 (two-tailed Student's t-test).

FIG. 18A illustrates the scheme illustrated the sensing of E. coli O157:H7 in agricultural water and the photograph demonstrated the fast-flow device driven by a syringe pump. FIG. 18B illustrates optical image and ARGB values of membranes treated by different concentrations of E. coli O157:H7 in spiked samples, sterilized Ag water, and Non-sterilized Agwater. FIG. 18C illustrates whole plate images of bacterial cultures upon exposure to non-sterilized agricultural water. Data are presented as mean±SD, with n=3 independent experiments.

FIG. 19A illustrates Optimization of (a) the reaction time between HRP and TMB substrate; FIG. 19B shows the concentration anti-E. coli O157:H7 antibodies used for immobilization: FIG. 19C shows the concentration of Anti-E. coli O157:H7 antibodies conjugated with HRP used as the secondary antibody in f-ELISA.

FIG. 20 illustrates Specificity of the assay. Images of the NHS@MF membranes with different treatments after the addition of TMB substrate: 100 μL Ab-E. coli (5 mg/L), 200 μL skimmed milk (SKM) (3%), 200 μL E. coli O157:H7 (10⁷ CFU/mL), and 100 μL Ab-E. coli-HRP (2 mg/L) were used accordingly. The bar diagram for the ARGB was observed from the images. Data are presented as mean±SD, with n=3 independent experiments.

FIG. 21 illustrates Optical image and ARGB values of membranes treated by different concentrations of E. coli O157:H7 in spiked milk samples. Data are presented as mean±SD, with n=3 independent experiments.

FIG. 22 illustrates Long-Term Stability Assessment. The Ab@NHS@MF membranes were prepared using 10% sucrose as a stabilizer followed by freeze-drying. They were stored at a consistent temperature of 4° C. and assessed over a period of 90 days. Data are presented as mean SD, with n=3 independent experiments.

### DETAILED DESCRIPTION

The present disclosure provides a three-dimensional (3-D) macroporous melamine foam (MF) membrane modified using an amine reactive coupling reagent to generate a secondary amine reactive group. The foam membrane is useful as a support for ELISA methods and sensor devices. The MF membrane can immobilize reagents for the methods and sensors disclosed herein. The use of a MF membrane significantly increases the binding capacity of biomolecules because the membrane provides 3D binding surfaces, which increases the surface area when compared to conventional 2D surface well plates.

In certain aspects, the amine reactive reagent is used to immobilize proteins. The amine reactive reagent or reactive group is a member selected from the group of an activated ester, a maleimide and a pyridyldithiol. The activated ester can be an NHS ester. In certain aspects, the activated ester is installed using a cross-linking agent such as N, N′-disuccinimidyl carbonate (DSC). In addition to DSC, other cross-linking reagents to install an NHS ester on the secondary amine include DSG (disuccinimidyl glutarate. DSS (disuccinimidyl suberate), BS3 (bis(sulfosuccinimidyl)suberate), BS(PEG)9 (PEGylated bis(sulfosuccinimidyl)suberate), DSP (dithiobis(succinimidyl propionate)), DTSSP (3,3′-dithiobis(sulfosuccinimidyl propionate)), EGS (ethylene glycol bis(succinimidyl succinate)) or a combination thereof.

| | | |
|---|---|---|
| DSC | DSC N,N′- disuccini- midyl carbonate | 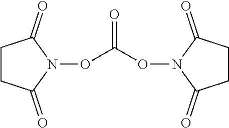 [SMILES: O=C(ON1C(=O)CCC1=O)ON1C(=O)CCC1=O] |
| DSG | DSG (disuccinimidyl glutarate) | 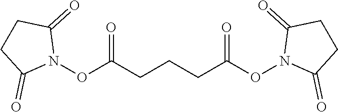 [SMILES: O=C(CCCC(=O)ON1C(=O)CCC1=O)ON1C(=O)CCC1=O] |
| | | [symbol] |
| DSS | DSS (disuccinimidyl suberate) | 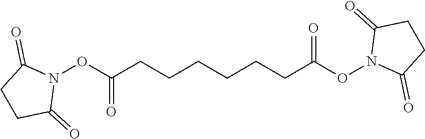 [SMILES: O=C(CCCCCCC(=O)ON1C(=O)CCC1=O)ON1C(=O)CCC1=O] |
| | | [symbol] |
| BS3 | BS3 (bis(sulfosuccini- midyl)suberate) | 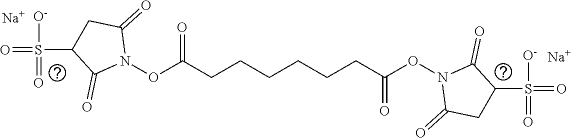 [SMILES: O=C(CCCCCCC(=O)ON1C(=O)CC(S(=O)(=O)[O-])C1=O)ON1C(=O)CC(S(=O)(=O)[O-])C1=O ; [Na+] ; [Na+]] |
| | | [symbol] |
| BS(PEG) 9 | BS(PEG)9 (PEGylated bis(sulfosuccini- midyl)suberate) | 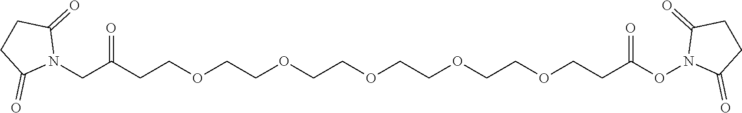 [SMILES: O=C(CCOCCOCCOCCOCCOCCC(=O)ON1C(=O)CCC1=O)CN1C(=O)CCC1=O] |
| | | [symbol] |
| | | 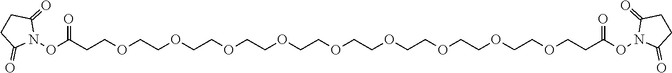 [SMILES: O=C(CCOCCOCCOCCOCCOCCOCCOCCOCCOCCC(=O)ON1C(=O)CCC1=O)ON1C(=O)CCC1=O] |
| | | [symbol] |
| DSP | DSP (dithio- bis(succini- midyl propionate)) | 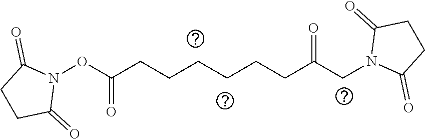 [SMILES: O=C(CCCCCCC(=O)ON1C(=O)CCC1=O)CN1C(=O)CCC1=O] |
| | | [symbol] |
| DTSSP | DTSSP (3,3′- dithiobis(sulfo- succini- midyl propionate)) | 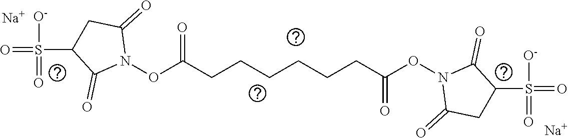 [SMILES: O=C(CCCCCCC(=O)ON1C(=O)CC(S(=O)(=O)[O-])C1=O)ON1C(=O)CC(S(=O)(=O)[O-])C1=O ; [Na+] ; [Na+]] |
| | | DTSSP |
| | | [symbol] |
| EGS | EGS (ethylene glycol bis(succinimidyl succinate)) | 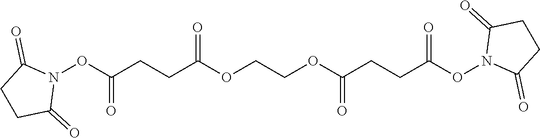 [SMILES: O=C(CCC(=O)ON1C(=O)CCC1=O)OCCOC(=O)CCC(=O)ON1C(=O)CCC1=O] |
| | | [symbol] |

[symbol]

indicates data missing or illegible when filed

In certain aspects, as shown above, the cross-linking agent can have different chain lengths to accommodate different analyte (e.g., protein) sizes. Bifunctional crosslinkers are reagents that contain two or more reactive groups which covalently attach via a spacer to, on the one hand, functional groups that are on proteins or other biomolecules and to MF on the other hand. The above homobifunctional crosslinking reagents have identical reactive groups so as to link an amine on MF to an amine on a protein. In addition to the hombifunctional crosslinking reagents above, heterobifunctional crosslinking reagents have different reactive groups such as amine-to-sulfhydryl groups such as NHS-maleimide or NHS-pyridyldithiol crosslinkers as shown below. These crosslinking reagents have reactive groups so as to link an amine on MF to a sulfhydryl on a protein. In addition, EDC activates carboxyl groups to conjugate to amino groups.

| | | |
|---|---|---|
| SMCC | Succinimidyl-4-(N- maleimidomethyl)cyclohexane-1- carboxylate (SMCC) i | 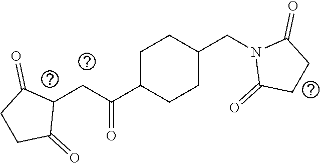 [SMILES: O=C(CC1C(=O)CCC1=O)C1CCC(CN2C(=O)CCC2=O)CC1] |
| | | [symbol] |
| Sulfo-SMCC | Sulfo-SMCC (sulfosuccinimidyl 4- (N-maleimidomethyl)cyclohexane- 1-carboxylate) | 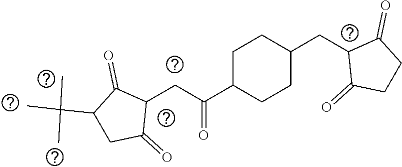 [SMILES: CC(C)(C)C1CC(=O)C(CC(=O)C2CCC(CC3C(=O)CCC3=O)CC2)C1=O] |
| | | [symbol] |
| SPDP | SPDP (succinimidyl 3-(2- pyridyldithio)propionate) | 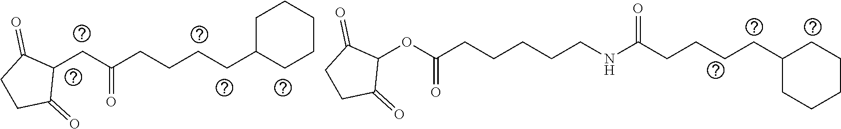 |
| | | [symbol] |
| EDC | 1-Ethyl-3-[3- dimethylaminopropyl]carbodiimide hydrochloride | 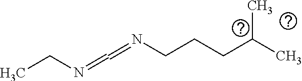 |

[symbol]

indicates data missing or illegible when filed

The chemically modified MF can be used as sensing materials for competitive, sandwich, indirect and direct ELISA sensing applications. For example, a SARS-CoV-2 spike protein, a transmembrane protein of SARS-CoV-2 virus, and chloramphenicol (CAP) were employed in this disclosure to illustrate applicability of the materials, or analytes that can be detected. The results show that the MF materials detect the SARS-CoV-2 spike protein receptor binding domain (SP-RBD) at 0.1 mg/L level with a limit of detection (LOD) at 0.047 mg/L, and chloramphenicol (CAP) at 1 ng/mL level for naked eyes and 0.096 ng/mL with the help of a smartphone such as an i-phone.

Melamine (MF) is an organic compound with the formula C₃H₆N₆. Melamine foam is commercially available, has a 1,3,5-triazine skeleton, and is made by reacting melamine with formaldehyde which yields secondary amine groups (see, Example 1).

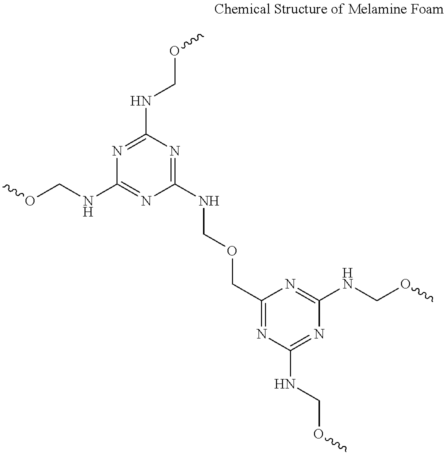

Advantageously, the secondary amine groups can be used directly to append a biomolecule or ligand, wherein the biomolecule is activated with an amine coupling group or alternatively, the melamine is modified to comprise an amine reactive group. The amine reactive group can then be used to couple a reagent such as a biomolecule. As such, the amine reactive group can be on the melamine foam or on the reagent (e.g., biomolecule).

Suitable amine reactive groups include, but are not limited to, an activated ester, a malimide or a pyridyldithiol. An activated ester can be installed in the MF with N, N′-disuccinimidyl carbonate (DSC).

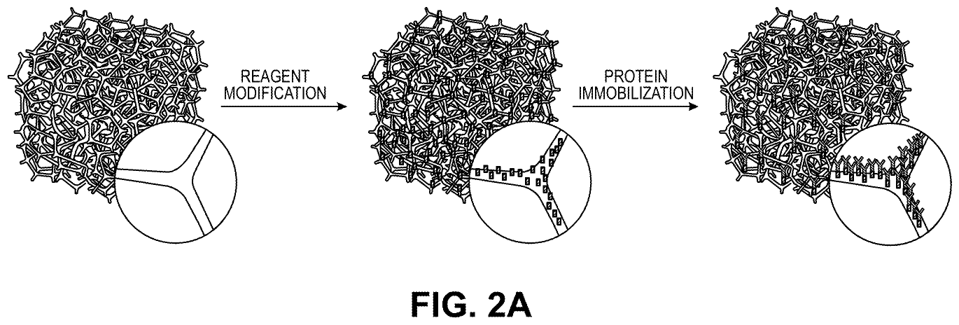

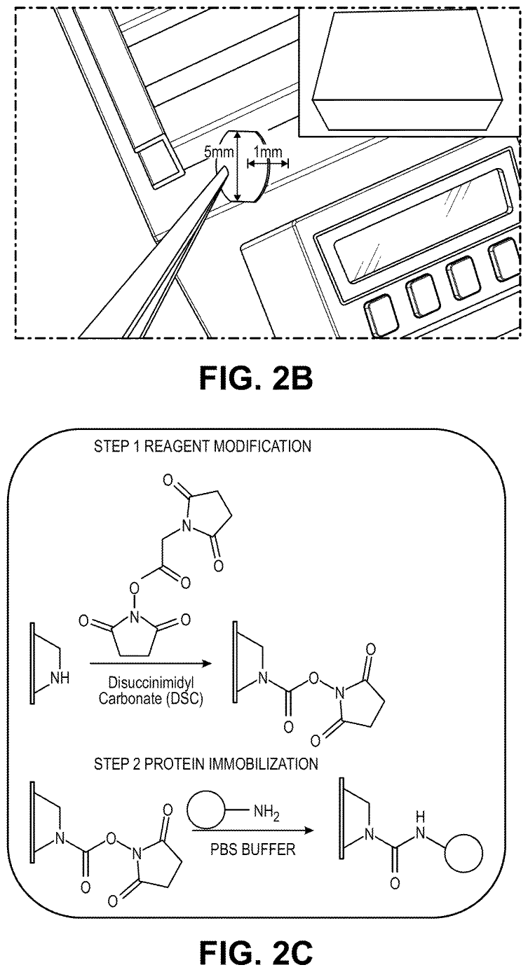

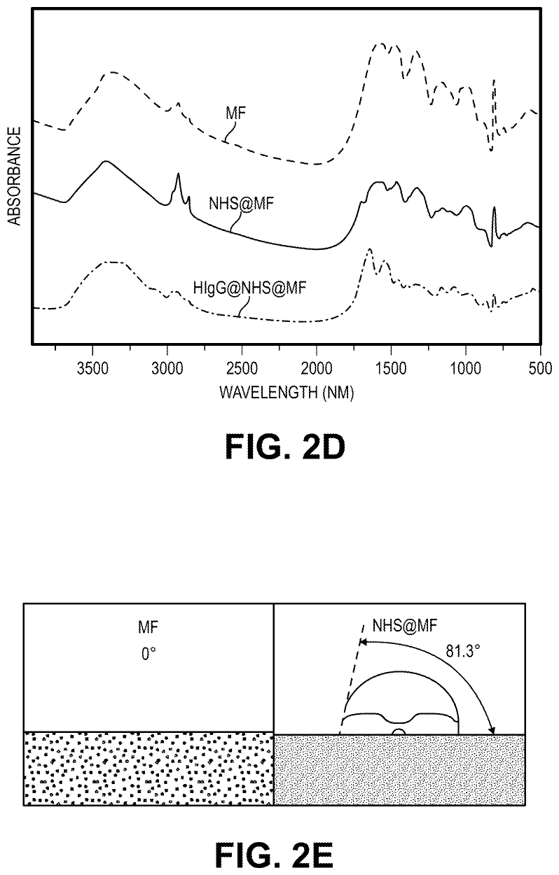

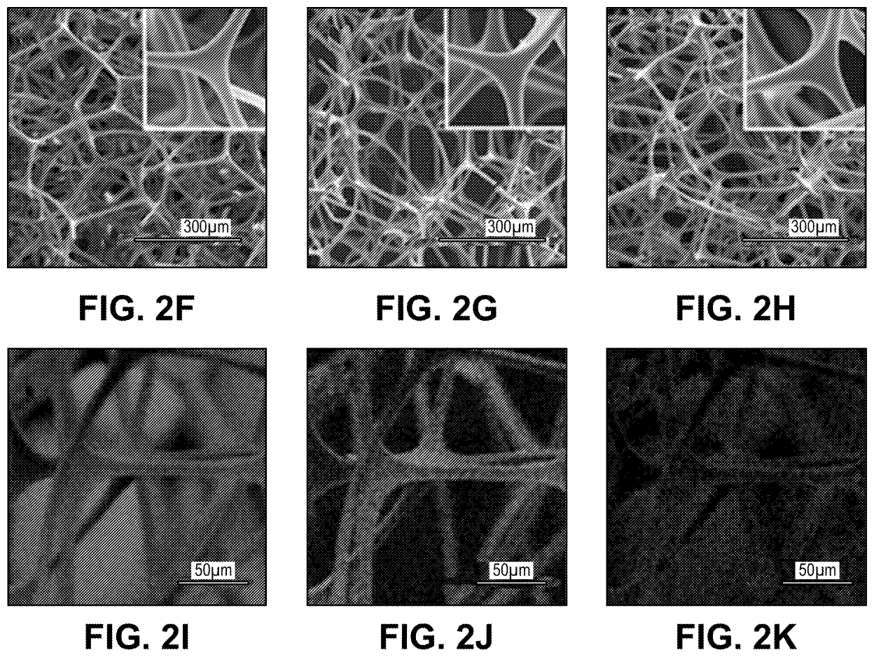

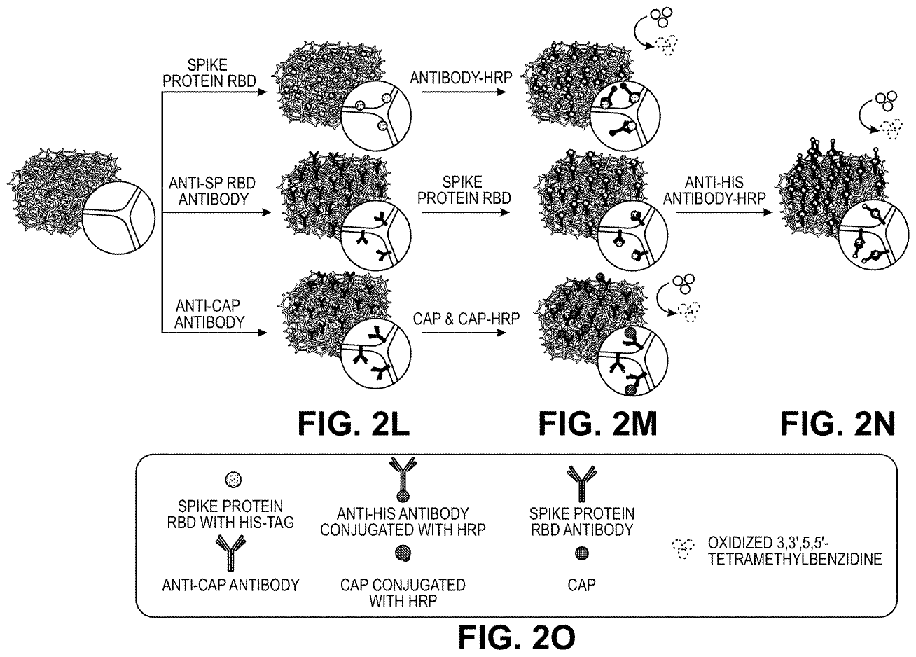

As shown in FIG. 2C, melamine foam is a framework structured material comprising active secondary amine groups. In one aspect, to covalently immobilize reagents, proteins, peptides or other biomolecules containing a primary amine onto MF, chemical modification is needed to activate the secondary amino groups on the material (FIG. 2A), which can be activated by for example, DSC to introduce NHS ester functional groups on the material (NHS@MF) for immobilization of primary amine biomolecules (e.g., proteins). The primary amine of the biomolecule can react with the NHS ester to form an amide bond.

When linking the secondary amine of the MF with an amine-containing reagent, ligand or biomolecule, the secondary amine is first converted to a more reactive form, e.g, a N-hydroxy succinimide (NHS) ester, by means of an activating reagent. The amine-containing ligand or biomolecule is then contacted with for example, the resulting activated acyl group to form an amide linkage. This reaction can be carried out in aqueous buffer with DMSO or DMF as an optional co-solvent. Alternatively, this reaction can be carried out in distilled water or in an aqueous buffer solution.

The secondary amine can be converted to an activated ester. An “activated ester” includes a derivative of a carboxyl group that is more susceptible to displacement by nucleophilic addition and elimination than an ethyl ester group (e.g., an NHS ester, a sulfo-NHS ester, a PAM ester, or a halophenyl ester). Representative carbonyl substituents of activated esters include succinimidyloxy (—OC₄H₄NO₂), sulfosuccinimidyloxy (—OC₄H₃NO₂SO₃H), -1-oxybenzotriazolyl (—OC₆H₄N₃); 4-sulfo-2,3,5,6-tetrafluorophenyl; or an aryloxy group that is optionally substituted one or more times by electron-withdrawing substituents such as nitro, fluoro, chloro, cyano, trifluoromethyl, or combinations thereof (e.g., pentafluorophenyloxy, or 2,3,5,6-tetrafluorophenyloxy). Preferred activated esters include succinimidyloxy, sulfosuccinimidyloxy, and 2,3,5,6-tetrafluorophenyloxy esters.

In certain aspects, to generate a melamine foam membrane comprising an amine reactive group, MF is immersed into a DSC modification solution. A DSC solution is prepared by dissolving DSC and triethylamine in 1,4 dioxane. The mixture is stirred for two hours at 70° C. The modified membranes (NHS@MF) can be thoroughly washed with 1,4-dioxane for 15 minutes twice and with acetone for 10 minutes and vacuum dried.

The modified MF can be used to attach reagents, biomolecules or ligands such as a protein, a peptide, a hormone, an antibody, an antigen, a hapten, or a carbohydrate. In certain aspects, the biomolecule or ligand comprises a primary amine, which can react with an activated ester.

The present disclosure provides various ELISA (Enzyme-linked immunosorbent Assay) formats using the modified melamine foams. In certain embodiments, the ELISA assay can be a sandwich ELISA, direct ELISA, indirect ELISA, or competitive ELISA assay. In general, a sandwich ELISA measures an analyte (e.g., antigen) between two layers of antibodies (a capture and a detection antibody). The target analyte contains at least two antigenic sites capable of binding to two antibodies. Monoclonal or polyclonal antibodies can be used as the capture and detection antibodies in sandwich ELISA systems.

For example, in certain instances, the present disclosure provides a sandwich ELISA method for determining the presence of an analyte in solution, the method comprising:
contacting a macroporous melamine foam membrane comprising an immobilized capture antibody with a sample containing an analyte, wherein the capture antibody is specific for an epitope of the analyte (e.g., such as an antigen) to form a captured analyte:
contacting the captured analyte with a detection antibody to form a sandwiched moiety; and detecting an output signal from the sandwiched moiety.

In certain instances, the detection antibody is labeled. In other instances, the detection antibody is unlabeled. In other instances, the assay further comprises a secondary enzyme-conjugated detection antibody. For example, the enzyme can be horseradish peroxidase. HRP substrates have been designed so that they generate a chemiluminescent, chromogenic, or fluorescent signal upon oxidation. The analyte can be a foodborne pathogen.

In certain instances, the present disclosure provides a direct ELISA method for determining the presence of an analyte, the method comprising:
contacting a macroporous melamine foam membrane comprising an immobilized analyte (e.g., antigen) with a sample comprising a capture antibody, wherein the capture antibody is specific for an epitope of the analyte (e.g., antigen) to form a captured analyte; and
detecting an output signal from the captured analyte.

In certain instances, in the direct ELISA, the analyte (e.g., antigen) is immobilized directly on the MF and a detection antibody binds to the analyte (e.g., antigen). In a direct ELISA only one antibody is used, wherein this single antibody is conjugated directly to a label (e.g., the detection enzyme). In a direct ELISA, the capture antibody comprises a label. The analyte can be a foodborne pathogen.

In certain other instances, the present disclosure provides a competitive ELISA method for determining the amount an analyte in sample, the method comprising:
contacting a macroporous melamine foam membrane comprising an immobilized capture antibody with a sample containing the analyte and a conjugated analyte, wherein the analyte competes with the conjugated analyte in the sample for the immobilized capture antibody to form a captured analyte; and
detecting an output signal from the captured analyte.

In certain other instances, the present disclosure provides competitive ELISA method for determining the amount a foodborne pathogen in a sample, the method comprising:
contacting a macroporous melamine foam membrane comprising an immobilized capture antibody with a sample containing the foodborne pathogen and a conjugated foodborne pathogen, wherein the conjugated foodborne pathogen competes with the foodborne pathogen in the sample for the capture antibody to form a captured analyte; and
detecting an output signal from the captured foodborne pathogen.

In certain instances, the capture antibody optionally comprises a label.

In certain instances, the conjugated antigen optionally comprises a label.

In certain instances, the conjugated foodborne pathogen optionally comprises a label.

In certain instances, the capture antibody comprises a label.

In certain instances, the conjugated antigen comprises a label.

In certain instances, the conjugated foodborne pathogen comprises a label.

In certain instances, the label comprises an enzyme.

In certain instances, the label comprises an enzyme. For example, the enzyme can be horseradish peroxidase. HRP substrates have been designed so that they generate a chemiluminescent, chromogenic, or fluorescent signal upon oxidation.

In certain instances, the higher the sample antigen concentration, the weaker the output signal, indicating that the signal output inversely correlates with the amount of antigen in the sample. The analyte can be a foodborne pathogen.

In alternative embodiments, the assays can be high throughput, multiplexed sensor or sensor system. The assays can be a nucleic acid based assay; an antibody based assay; an enzyme based assay; a chemical based assay: a hybridization; a molecular beacon: an aptamer; a real-time fluorescent sensor; an ELISA: a sandwich based assay; an immunostaining assay; an antibody capture assay; a secondary antibody amplification assay; a proximity ligation based assay; an enzyme based assay comprising use of PCR, RT-PCR. RCA, loop-mediated isothermal amplification (LAMP), nicking, strand displacement and/or an exponential isothermal amplification; or any combination thereof. Other assays include, but are not limited to, an immunoassay (e.g., radioimmunoassay, Western blotting, immunoprecipitation, immunostaining, immunofluorescence, and enzyme-linked immunosorbent assay (ELISA) (e.g., sandwich ELISA, indirect ELISA, competitive ELISA), magnetic immunoassay, and the like).

In certain instances, the assay methods and systems of the disclosure can analyze a biological sample or biomolecule which can comprise a biopsy, blood, serum, saliva, tear, stool, urine or CSF sample from an individual or a patient. In alternative embodiments, methods and systems of the disclosure can analyze any sample obtained from a food, water, soil, a crop or an air source.

In certain instance, the present disclosure provides methods and biosensors for the detection of foodborne pathogens. Foodborne illness is caused by consuming contaminated foods or beverages. Most foodborne diseases are infections caused by a variety of pathogens such as bacteria, viruses, and parasites. Also, foodborne diseases are often associated with the consumption of raw or undercooked foods such as seafood, meat, and poultry. It is essential to analyze the food for the presence of foodborne pathogens in order to ensure a safe food supply and to minimize the occurrence of foodborne diseases.

In certain aspects, in practicing methods and systems of the invention, the samples can be directly assayed with no or minimal (e.g., dilution) concentration processing. Standard established biological sample preparation processes including dilution, purification, enrichment, extraction, centrifugation, magnetic bead assays, and washing steps, although not required, can be integrated into assays, methods and systems of the disclosure.

The systems and methods can be used in monitoring chemical pesticides, herbicides, and other environmental pollutants. Portable devices and sensors as described herein can be used in monitoring foodborne pathogens, chemical pesticide residues in food crops, and other applications with such sensors working with small amounts of sample. Pesticide determination is important due to the fact that the chemicals exhibit high acute toxicity and can cause long-term damage to the environment and human lives even at trace levels.

In certain aspects, the present disclosure provides sensors with one or more of the following attributes, which include, a personal-use, naked eye readable, low cost, highly sensitive, and selective biosensors for rapid detection of environmental toxicants are relevant for many applications. The highly sensitive and naked eye distinguishable colorimetric sensors can be manufactured by using commercially available melamine foam (MF) as basic sensing materials. The sensors can be for instant detection and/or volume-responsive simultaneous detection of multiple targets in fluid systems.

In certain other aspects, the biosensors and methods are useful for the detection of foodborne pathogens such as bacteria or microbes. Foodborne illnesses are caused by consuming contaminated foods or beverages. Most foodborne diseases are infections caused by a variety of pathogens such as bacteria, viruses, fungi, and parasites. The biosensors and methods disclosed herein are useful for detecting such pathogens and have one or more of the following attributes, high specificity (e.g., detecting only the bacteria of interest), high sensitivity (e.g., capable of detecting as low levels of bacteria) short time-to-results (e.g., minutes to hours), great operational simplicity (e.g., use of a smartphone for detection) and cost effectiveness.

The MF possesses a unique reticulated three-dimensional (3D) macroporous framework structure enabling rapid mass transfer of large biomolecules through the structures in all directions, ensuring easy access of numerous active binding sites of the chemically modified framework to the proteins and target molecules, and subsequently providing significantly increased sensitive and volume-responsive detection of target molecules in flow-through sensor systems. The MF can be used as a substrate for direct, sandwich, and competitive ELISA tests.

An additive and simultaneous detection of two targets (multiplex) in one system is achieved by using different layers of the sensor materials in a flow-through filtering device. The biosensors significantly improve the sensitivity and broaden the applications of ELISA in rapid detections of trace amounts of toxicants in liquid and aerosol systems.

The MF sensors of the present disclosure have been prepared using various methods and have exceptional sensitivity. The sensor materials have one or more of the following characteristics: 1) ultra-sensitive to low concentrations of bacteria in small or large samples, especially for fluids; 2) filter-like sensor materials that can be varied in different thickness and shapes suitable for various applications of food drink fluids and wastewater; 3) can be additive for detection different targets simultaneously in one sample; 4) volume responsive signals with signal intensity linearly relating to volumes; or 5) suitable for low concentrations of bacteria cells in large volume samples.

In certain aspects, the sensors and methods of the present disclosure can be used to detect foodborne pathogens such as bacteria. Bacteria related food poisoning is common. More than 90 percent of the cases of food poisoning each year are caused by one or more of the following Staphylococcus aureus, Salmonella, Clostridium perfringens, Campylobacter, Listeria monocytogenes, Vibrio parahaemolyticus, Bacillus cereus, or Entero-pathogenic Escherichia coli. These bacteria are commonly found on many raw foods. Normally a large number of food-poisoning bacteria must be present to cause illness. Therefore, illness can be prevented by detection.

Other pathogens that cause food poisoning include Acinetobacter spp., Bacillus subtilis, Citrobacter koseri, C. freundii, Clostridium difficile, Enterobacter sakazakii, E. cloacae, Escherichia coli O157:H7, Klebsiella oxytoca, K. pneumoniae, Shigella sonnei, Vibrio cholerae and Yersinia pestis.

Shiga toxin producing E. coli O157:H7 (STEC) is a major pathogen which is responsible for foodborne outbreaks. The outbreaks can be due to different subtypes of E. coli O157:H7, termed as enterohaemorrhagic E. coli (EHEC) which has the characteristics of both verotoxigenic E. coli and of a lesser known diarrhoeagenic enteroaggregative E. coli.

Foodborne pathogens can lead to serious outbreaks, which can lead to the spread of disease, more so in infants and aged individuals. Rapid detection becomes important to contain the spread of the pathogen before it leads to a serious outbreak.

In certain instances, the immunoassays disclosed herein can use polyclonal or monoclonal antibodies. The ELISAs have high sensitivity and high specificity, which reduce false positives and false negatives. The substrates bind to the respective conjugates specifically and develop coloration which can be read on a smartphone. The color change is visible to the naked eye. One substrate used is 2,2′-azino-bis(3-ethylbenzthiazoline-6-sulphonic acid). Tetramethylbenzidine is another substrate that is commonly used in ELISA. It binds to horse radish peroxidase (HRP). The coloration may develop gradually. Another commonly used substrate is p-nitrophenyl phosphate (pNPP).

To overcome high detection limits, it is possible to include enrichment steps for the detection of pathogens in food products. In certain aspects, with an enrichment step, a simple and rapid detection is possible with simultaneous enrichment and optical detection. The principle of this method is culture/capture/measure.

The detection of foodborne pathogens by immunological-based methods is based on antibody-antigen interactions, whereby a particular antibody will bind to its specific antigen. There are different types of enzymes that can be used in ELISA, which include horseradish peroxidase (HRP), alkaline phosphatase and beta-galactosidase. In certain instances, a liquid sample of an enriched sample is placed on a the reagent strip that contains all the required reagents in a ready-to-use format.

In an exemplary multiplex assay, a competitive ELISA assay was used to achieve simultaneous multiple on-site targets detection. First, 100 μL 25 mg/L Ab-CAP and Ab-CPS were added into two different groups of NHS@MF membranes separately. Ab-CAP is an antibody against chloramphenicol (CAP). Ab-CPS is an antibody against Chlorpyrifos (CPS).

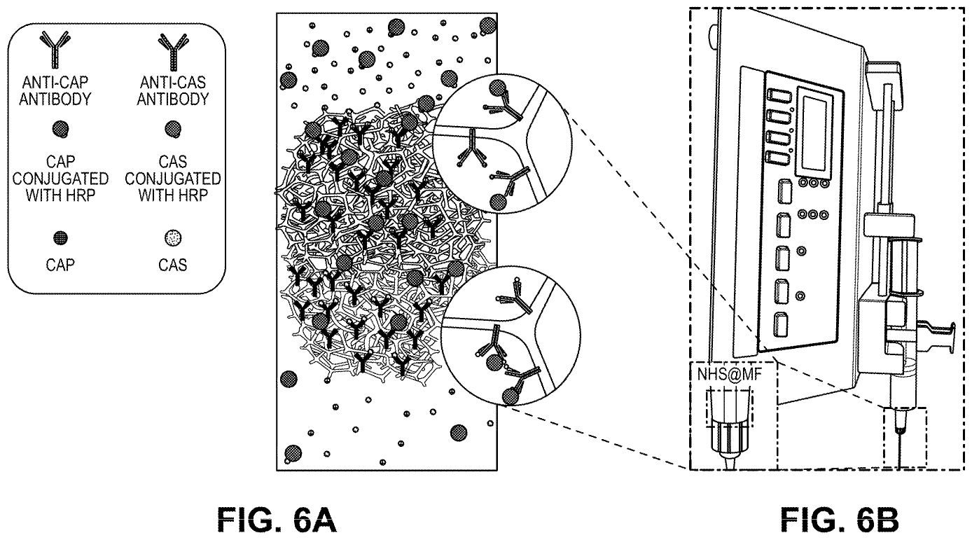

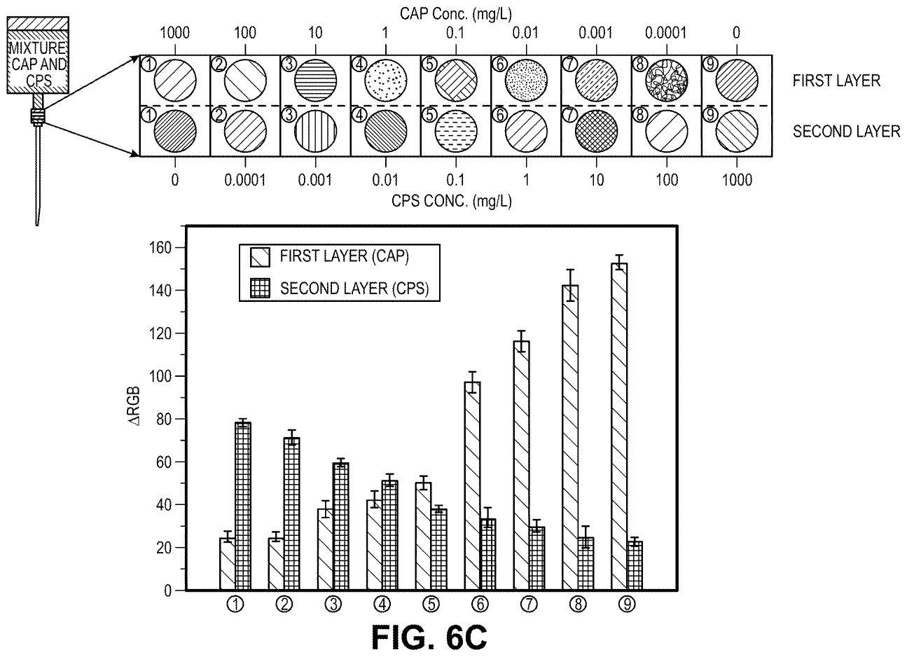

Both groups of the membranes were exposed to 3% BSA to block the remaining active sites. After blocking, one membrane was selected from the Ab-CAP immobilized group and one membrane was selected from the Ab-CPS immobilized group and placed them into a 20 mL syringe needle as shown in FIG. 6B.

The order of different layers should be remembered. Then 2 mL of a mixture of CAP and CPS in specific concentrations, and 40 μL of mixture solution of CAP-HRP and CPS-HRP in a concentration of 100 mg/L each are filled into the syringe. The filtration flow rate was controlled by a SyringeONE programmable syringe pump (NewEra Instruments, USA) with a flow rate of 15 mL/h. Then the column is successively washed with 20 mL tween-20 (0.05%) and PBS buffer. The membranes mounted in syringe needles are collected separately, and 25 μL of TMB substrate (ThermoFisher) is then applied to the membranes. By analyzing the colorimetric signals obtained from the picture of a smartphone (e.g., iPhone 8), simultaneous multiple on-site targets detection can be achieved.

Detectable labels that find use in practicing the subject methods include, but are not limited to, a fluorophore, a chromophore, an enzyme, a linker molecule, a biotin molecule, an electron donor, an electron acceptor, a dye, a metal, or a radionuclide. Detectable labels may be selected from a variety of such labels, including chromophores, fluorophores, fluorochromes, enzymes (e.g., horseradish peroxidase or other peroxidases), linker molecules or other moieties or compounds which either emit a detectable signal (e.g., fluorescence, color) or emit a detectable signal after exposure of the label to its substrate. Various detectable label/substrate pairs (e.g., horseradish peroxidase/diaminobenzidine, biotin/streptavidin, luciferase/luciferin), methods for labeling antibodies, and methods for using labeled secondary antibodies to detect an antigen are well known in the art.

According to certain embodiments, the detectably labeled antibody is fluorescently-labeled and includes a fluorophore selected from indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, Alexa Fluor 700, JOE, Lissamine, Rhodamine Green, BODIPY, fluorescein isothiocyanate (FITC), carboxy-fluorescein (FAM), Allophycocyanin (APC), phycoerythrin (PE), rhodamine, dichlororhodamine (dRhodamine), carboxy tetramethylrhodamine (TAMRA), carboxy-X-rhodamine (ROX), LIZ, VIC, NED, PET, SYBR, PicoGreen, and RiboGreen.

When the detectably labeled antibody is fluorescently-labeled, the detecting may include detecting one or more fluorescence emissions. The fluorescence emission(s) may be detected in any useful format. In certain aspects, the detecting includes viewing the color or fluoresce with the naked eye or help with a mobile phone.

Optionally, a control or standard is included in an assay according to aspects of the present invention. The terms “control” and “standard” are familiar to those of ordinary skill in the art and refer to any control or standard that can be used for comparison. The control or standard may be determined prior to the analyte assay, in parallel, simultaneously, in a multiplex assay or other assay format. A control or standard can be a negative control and/or a positive control.

According to aspects of this disclosure, immunoassay kits for detecting an analyte in a sample are provided which include one or more antibodies or antigen binding fragments which specifically bind to the analyte or a antigen to be immobilized.

One or more auxiliary components are optionally included in such kits, such as a control such as one or more conjugates, membrane foam, a secondary antibody, one or more reaction vessels, a buffer, diluent or a reconstituting agent.

Alkaline phosphatase, horseradish peroxidase (HRP) and β-galactosidase are examples of enzyme labels (and at the same time optical labels) which catalyze the formation of chromogenic reaction products. For example, the main colorimetric substrate for HRP is TMB (3, 3′, 5, 5′-tetramethylbenzidine). TMB produces a deep blue color during the enzymatic degradation of hydrogen peroxide by HRP, and the addition of an appropriate stop solution gives a clear yellow color, which absorbs at 450 nm.

By analyzing the colorimetric signals obtained from the picture of a smartphone (e.g., iPhone 8) of the yellow color, simultaneous multiple on-site targets detection is achieved.

In one exemplary process, when TMB is added to the membrane foams, the membranes were placed in an LED lightbox (E mart), and images were captured through the smartphone camera. The R channel value of the area of interest is obtained by using Photoshop software from Adobe®.

The red channel (R) values from RGB values represent the color intensity. Here, the red channel intensity change can be represented by the ARGB value, which was obtained by the RGB value difference between the white background and each membrane, from the equation:

$$Δ RGB = RGBbackground - RGBmembranes$$

The ARGB value is color intensity.

Embodiments of the disclosure including sensors and methods are illustrated in the following examples. These examples are provided for illustrative purposes and are not considered limitations on the scope of inventive compositions and methods.

### EXAMPLES

### Example 1

### 1.1 Melamine Foam

By “melamine foam” (MF) it is meant a melamine-formaldehyde resin foam.

A suitable melamine-formaldehyde resin foam raw material is commercially available under the trade name Basotect® from BASF.

As described in U.S. Pat. No. 8,440,604, the term “melamine foam” can be prepared by blending major starting materials of melamine and formaldehyde, or a precursor thereof, with a blowing agent, a catalyst and an emulsifier, injecting the resultant mixture into a mold, and making the reaction mixture generate heat through a proper means such as heating or irradiation with electromagnetic wave to cause foaming and curing. The molar ratio of melamine to formaldehyde (i.e., melamine:formaldehyde) for producing the precursor is preferably about 1:1.5 to about 1:4, particularly preferably about 1:2 to about 1:3.5 in melamine:formaldehyde. In addition, number average molecular weight of the precursor is preferably about 200 to about 1,000, particularly preferably about 200 to about 400. Additionally, formalin, which is an aqueous solution of formaldehyde, is usually used as formaldehyde.

As monomers for producing the precursor, the following various monomers may be used in an amount of 5 about 0 parts by weight (hereinafter abbreviated as “parts”) or less, particularly about 20 parts by weight or less, per about 100 parts by weight of the sum of melamine and formaldehyde in addition to melamine and formaldehyde. As other monomers corresponding to melamine, there may be used C₁₋₅ alkyl-substituted melamines such as methylolmelamine, methylmethylolmelamine and methylbutylolmelamine, urea, urethane, carbonic acid amides, dicyandiamide, guanidine, sulfurylamides, sulphonic acid amides, aliphatic amines, phenols and the derivatives thereof. As aldehydes, there may be used acetaldehyde, trimethylol acetaldehyde, acrolein, benzaldehyde, furfurol, glyoxal, phthalaldehyde, terephthalaldehyde, etc.

As the blowing agent, there may be used pentane, trichlorofluoromethane, trichlorotrifluoroethane, etc. However, use of so-called Fleons® such as trichlorofluoromethane is regulated from the point of view of environmental problems, thus not being preferred. On the other hand, pentane is preferred in that it easily provides a foam when used even in a small amount but, since it has a volatile flammability, it requires sufficient care in its handling. Further, as the catalyst, formic acid is commonly used and, as the emulsifier, anionic surfactants such as sodium sulfonate may be used.

The amount of the electromagnetic wave to be irradiated for accelerating the curing reaction of the reaction mixtures is preferably adjusted to be about 500 to about 1,000 kW, particularly about 600 to about 800 kW, in electric power consumption based on 1 kg of an aqueous formaldehyde solution charged in the mold. In case when this electric power consumption is insufficient, there results an insufficient foaming, leading to production of a cured product with a high density. On the other hand, in case when the electric power consumption is excessive, the pressure upon foaming becomes seriously high, leading to serious exhaustion of the mold and even the possibility of explosion. Thus, electric power consumption outside the range is not preferred.

### 2.1 Chemicals and Materials

N, N′-disuccinimidyl carbonate (DSC), triethylamine (TEA), 1,4-dioxane, acetone, phosphate-buffered saline (PBS), chlorpyrifos polyclonal antibody, pierce BCA protein assay kit, and 96-well plates were purchased from ThermoFisher Scientific. The SARS-CoV-2 spike protein receptor-binding domain with C-Histag (SP-RBD) was purchased from Sino Biological. Chloramphenicol (CAP), Chlorpyrifos (CPS) solution, human immunoglobulin G (HIgG), and fluorescein isothiocyanate (FITC) linked dextran (FITC-Dextran, 40 KDa) were purchased from Sigma-Aldrich. Anti-CAP antibody (Ab-CAP) and CAP-labelled horseradish peroxidase (CAP-HRP) were purchased from Abcam (Cambridge, MA, USA). Anti-chlorpyrifos monoclonal antibody (Ab-CPS) and chlorpyrifos (HRP) (CPS-HRP) were purchased from CD Creative Diagnostics. 6×-His Tag Monoclonal Antibody (HIS.H8) Alexa Fluor 647 (Ab-HIS-647). 6×-His Tag Monoclonal Antibody (HIS.H8) HRP (Ab-HIS-HRP) and SARS-CoV-2 Spike Protein (RBD) Recombinant Human Monoclonal Antibody (Ab-SP) were purchased from Thermo Fisher. Melamine foams were purchased from Swisstek (Swisstek Manufacturer).

### 2.2 Measurement of Diffusion of Biomolecules in MF

Diffusions of dextran (40 KDa) and Human IgG (150 KDa) in the melamine foam (MF) of different thicknesses were measured by using a side-by-side diffusion chamber (PermeGear Co.), which consists of two 3.4 mL chambers (donator chamber and receptor chamber) with a 9 mm orifice. MF membranes with different thicknesses (1 mm, 2 mm, 3 mm) were placed between the two chambers separately, and the chambers were tightly sealed and placed in a water bath to maintain the temperature at 25° C. To prewet the membranes. 3 mL of a PBS solution was added to each chamber individually. Then, 0.3 mL of a solution (10 g/L of Dextran-FITC or HIgG in PBS buffer was injected into the donor chamber after 15 min. Throughout the tests, stirring bars were placed in both chambers at a speed of 750 rpm. At regular time intervals, 50 μL of the sample solution was taken from each chamber and replaced with the same amount of PBS buffer solution for 15 min. The concentrations of FITC-Dextran and HIgG can be obtained with a microplate reader (SpectraMax® iD3 multi-Mode) according to calibration curves. Then, the protein concentration in the receptor chamber at increasing time periods can be utilized to determine diffusion properties of the biomolecules through the MF membranes.

### 2.3 Modification of Melamine Foam Membranes

0.2 Gram of MF in 1 mm thick slices and 5 mm diameter circular membranes (FIG. 2B) were immersed into a DSC modification solution (prepared by dissolving 5 g DSC and 0.4 g TEA in 100 mL of 1,4 dioxane). The mixture was stirred for two hours at 70° C. The modified membranes (NHS@MF) were thoroughly washed with 1,4-dioxane for 15 minutes twice and with acetone for 10 minutes and vacuum dried.

### 2.4 Characterizations

The structures and morphologies of the MF before and after the modification were observed by a scanning electron microscope (Quattro ESEM, Thermo Scientific). The thickness of the MF membranes was measured through an electronic micrometer thickness gauge (Neoteck). For measuring the NHS amount in a membrane, the NHS@MF membrane was dried in a vacuum oven for 10 min after the modification experiment. Then, the membrane was placed in 1 mL of a working solution following the BCA protocol, where the working solution was prepared by mixing 50-parts bicinchoninic acid (BCA) reagent A with 1-part Cu₂SO₄ reagent B. The NHS amount can be obtained with a micro-plate reader by calibration curves.

### 2.5 Immobilization of Proteins

The chemically modified MF membranes (NHS@MF) were immersed into an antibody (50 mg/L) (Alexa Fluor™ 647 conjugate) solution (200 uL) for 30 min and were washed several times using the PBS buffer before following measurements. A confocal microscope (FV 1000 system, Olympus America) was used to observe the distribution of immobilized protein on the membranes. FTIR was employed to characterize the membrane before and after the modification and immobilization following the protocols. BCA was used to determine the concentration of proteins that are covalently immobilized on MF membranes by calibration curves.

### 2.6 Immunoassays Based on NHS@MF: Direct ELISA, Sandwich ELISA, and Competitive ELISA

Direct and Sandwich ELISA assays were used to detect a SARS-CoV-2 spike protein receptor-binding domain (SP-RBD). For direct ELISA, 100 μL varied concentrations (ranging between 0 to 100 mg/L) of the SP-RBD were added to the NHS@MF membranes, and an incubation lasted for 30 min under gentle agitation. Then the membrane was exposed to 3% BSA (200 uL) to block the remaining active sites. Subsequently, 100 μL of 1 mg/L Ab-HIS-HRP was added to each membrane. After 20 min, the membranes were first washed with tween-20 (0.05%) and then washed with PBS buffer and dried in air. 25 μL of TMB substrate (ThermoFisher) was then applied onto the membranes, and membranes were placed in an LED lightbox (E mart). The colorimetric signal from the interaction between HRP and TMB substrate was captured by a smartphone (iPhone 8) and analyzed using a Photoshop (Adobe) software. To take pictures of each result, the smartphone was placed over membranes at a fixed distance of 50 cm. For Sandwich ELISA, 100 μL of the 5 mg/L Ab-SP was added to the membrane platform and incubated for 30 min. Then the membrane was exposed to 200 μL of 3% skim milk to block the remaining active sites. After blocking, 100 μL varied concentrations (ranging between 0 to 100 mg/L) of SP-RBD were added to the NHS@MF membranes, and the incubation lasted for 30 min under gentle agitation. Subsequently, 100 μL of 1 mg/L Ab-HIS-HRP was added to each membrane. After 20 min, the membranes were first washed with tween-20 (0.05%) and then washed with PBS buffer and dried in air. To obtain the outcome of colorimetric signals, the following steps are the same as in the direct ELISA.

A competitive ELISA assay was used to detect chloramphenicol (CAP), an antibiotic banned in use in USA but is still used in other countries. First, 100 μL of the 25 mg/L Ab-CAP was added to the membranes and incubated for 30 min. Then, 50 μL varied concentrations (ranging between 0 to 100 mg/L) of CAP were mixed with 50 μL of 2 mg/L CAP-HRP conjugate, and the 100 μL of the mixed solution was then added to each membrane. After 20 min, the membranes were first washed with tween-20 (0.05%) and then washed with PBS buffer, and lastly dried in air. The subsequent experimental steps are the same as the first two experiments. The red channel values (R value) could be read through a Photoshop's color histogram. The R values were correlated to the concentration of analytes. To further investigate the impact of the sample volume in different types of immunoassays based on the material, the varied volumes of samples (100 μL, 500 μL, 1 mL. 2 mL) were applied to each experiment. In this study, except for addition of varied volumes of analytes, the rest steps followed the same protocols as we mentioned above. The sample size of all experiments is 5.

### 2.7 Simultaneous Detection of Multiple Targets

A competitive ELISA assay was used to achieve simultaneous multiple on-site targets detection. First, 100 μL 25 mg/L Ab-CAP and Ab-CPS were added into two different groups of NHS@MF membranes separately. Then both groups of the membranes were exposed to 3% BSA to block the remaining active sites. After blocking, we selected one membrane from the Ab-CAP immobilized group and one membrane from the Ab-CPS immobilized group and placed them into a 20 mL syringe needle as shown in FIG. 6B. The order of different layers should be remembered. Then 2 mL of a mixture of CAP and CPS in specific concentrations, and 40 μL of mixture solution of CAP-HRP and CPS-HRP in a concentration of 100 mg/L each were filled into the syringe. The filtration flow rate was controlled by a SyringeONE programmable syringe pump (NewEra Instruments, USA) with a flow rate of 15 mL/h. Then the column was successively washed with 20 mL tween-20 (0.05%) and PBS buffer. The membranes mounted in syringe needles were collected separately, and 25 μL of TMB substrate (ThermoFisher) was then applied to the membranes. By analyzing the colorimetric signals obtained from the picture of a smartphone (iPhone 8), simultaneous multiple on-site targets detection can be achieved.

### 2.10 Colorimetric Data Processing

When TMB was added to the membranes, the membranes were placed in an LED lightbox (E mart), and images were captured through the smartphone camera. The R channel value of the area of interest was obtained by using the Photoshop software.

The red channel (R) values from RGB values represent the color intensity. Here, the red channel intensity change could be represented by ARGB value, which was obtained by the RGB value difference between the white background and each membrane, as the equation of

$$Δ RGB = RGBbackground - RGBmembranes$$

### 3. Results and Discussion

### 3.1 Molecule Diffusion in Melamine Foam Membranes

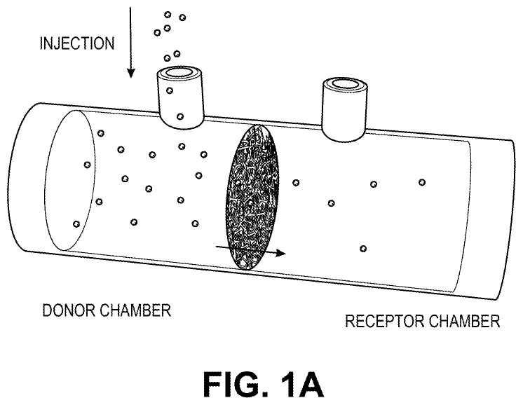

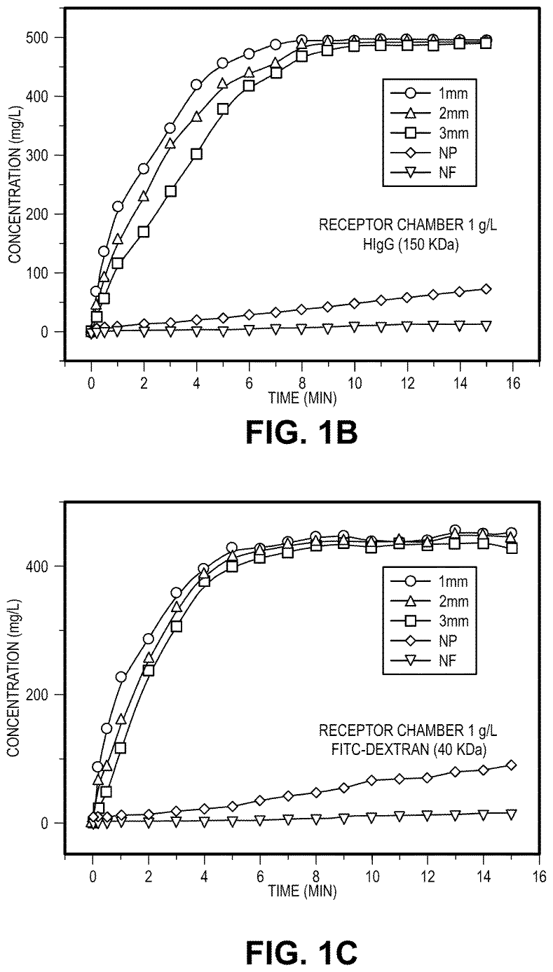

Diffusion of large biomolecules in electrospun microporous and nanofibrous membranes was proven heterogeneous and slow in vertical directions due to the fact of layered nanofibrous mats and reduced effective pores. The reticulated MF materials possess unique three dimensional macroporous structure and should allow large biomolecules to penetrate through with less resistance. A side-by-side diffusion chamber was employed to measure biomolecule transport in the MF membranes (FIG. 1A). FITC-dextran (40 KDa) and HIgG were employed as sample biomolecules to study their diffusion behaviors through the MF membranes because they had similar sizes as immunoglobulin (~150 KDa) and horseradish peroxidase (HRP) (~40 KDa respectively, which have been widely used in applications of immunoassays. The plots of concentrations of HIgG in the receiver chamber versus diffusion times are shown in FIG. 1B for MF membranes in varied thicknesses of 1 mm to 3 mm. With the thickness increase of the membranes from 1 mm to 3 mm, the diffusion times of IHgG to reach the steady-state diffusion slightly rose from 8 min to 10 min. While the FITC-dextran, compared with the diffusion behavior of HIgG through the membrane with the same thickness, needed 6 min, 7 min, and 8 min to reach the steady state diffusion pattern for 1 mm, 2 mm, and 3 mm membranes, respectively (FIG. 1C). The difference of the biomolecules in molecular size determines the difference of the diffusion rates, with larger molecules leading to slower diffusion through the membranes. However, compared to the diffusion efficiency of the large biomolecules in nanofibrous membranes (PVA-co-PE), which required hours to reach the steady-state of diffusion, the thicknesses of membranes and sizes of molecules did not show any significant impact and can be ignored as long as the time of interaction between the MF and substrate is longer than 10 min. In conclusion, the open framework structure, high porosity, and large pore size of the MF allow large biomolecules to penetrate through the membranes without mass transfer resistance.

### 3.2 Reagent Modification and Protein Immobilization

Melamine foam is a framework structured material consisting of active secondary amine groups. To covalently immobilize proteins on MF, chemical modification is needed to activate the secondary amino groups on the material (FIG. 2A), which could be activated by DSC to introduce the NHS functional groups on the material (NHS@MF) for immobilization of proteins, and the reactions of reagent modification and protein immobilization are shown in FIG. 2C. Fourier-transform infrared spectroscopy (FTIR) proved successful incorporations of the reactive groups (NHS) and immobilization of the protein based on carbonate peak of NHS at 1730 cm-1 and amideIpeak at 1625 cm-1 (FIG. 2D). DSC reagent provided an improved hydrophilic effect on its modification of PVA-co-PE nanofibrous membranes. However, as shown in FIG. 1E, the water contact angle of pristine MF is 0°, and the liquid drop completely spreads out on the solid surface. But the water contact angle of the DSC modified MF (NHS@MF) is 81.3°, meaning that the membranes become less hydrophilic after the reaction with DSC. Despite the decrease of hydrophilicity, the liquid drop will completely spread out on the modified membranes after around 40 seconds and the NHS@MF still retains the ideal hydrophilicity. The hydrophilicity of the MF is important in serving as a solid media for biosensors because the hydrophilic surface of the material can reduce non-specific protein adsorption and promotes protein diffusion through the membrane, making elusion of any unbonded substances easily and completely in each step. The SEM images shown in FIG. 2F-H indicate that before and after the modification and immobilization, the morphology of the MF framework structures intact with a pore size of around 100 μm and a fiber diameter of around 5 μm.

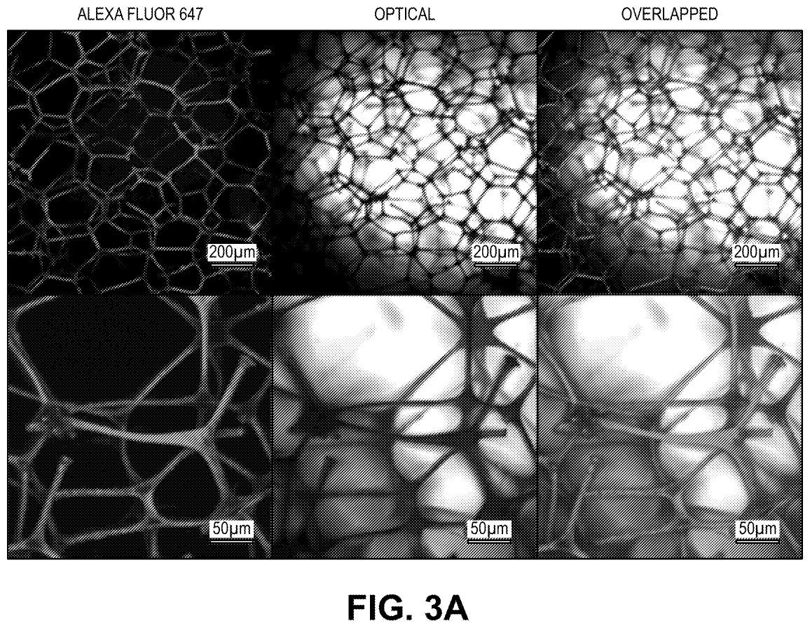

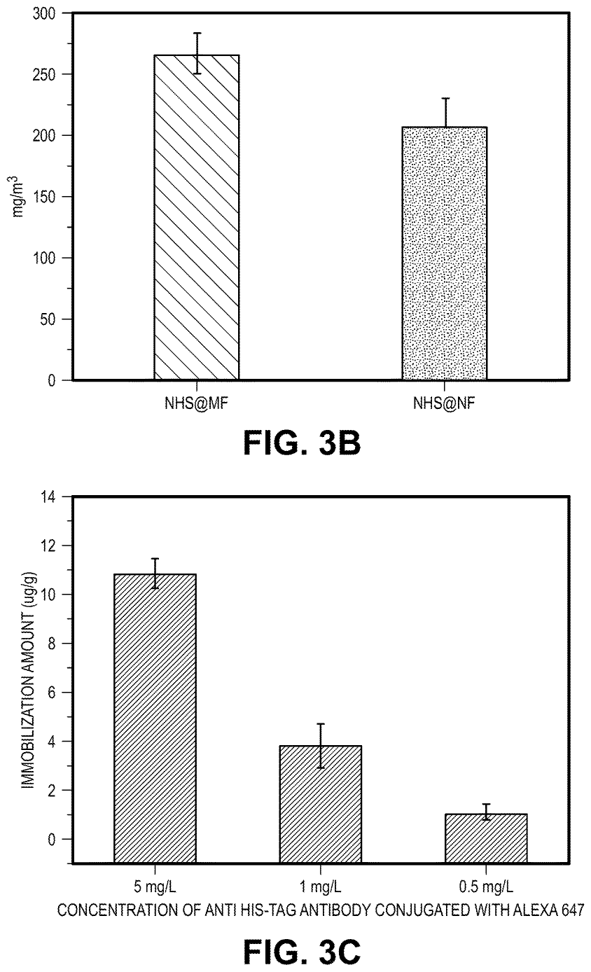

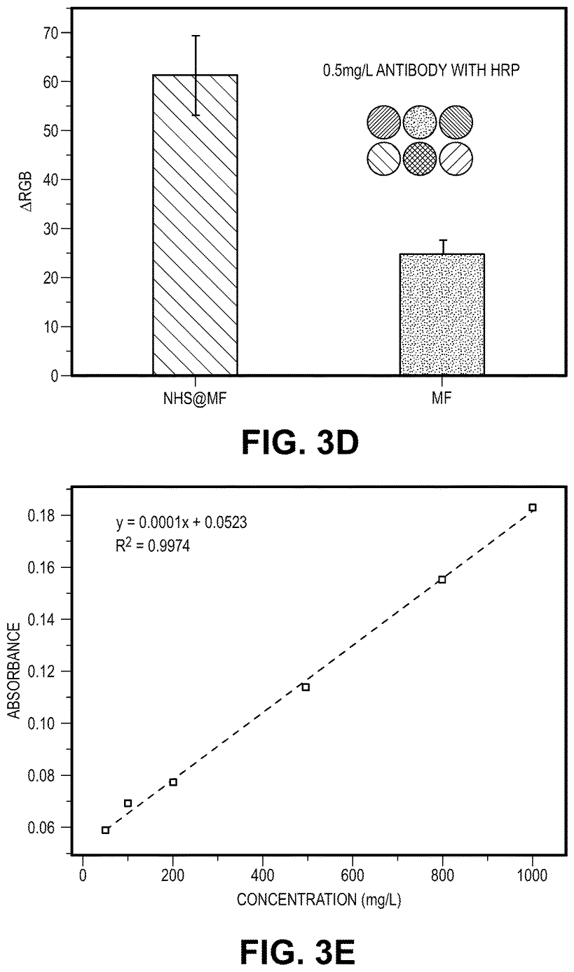

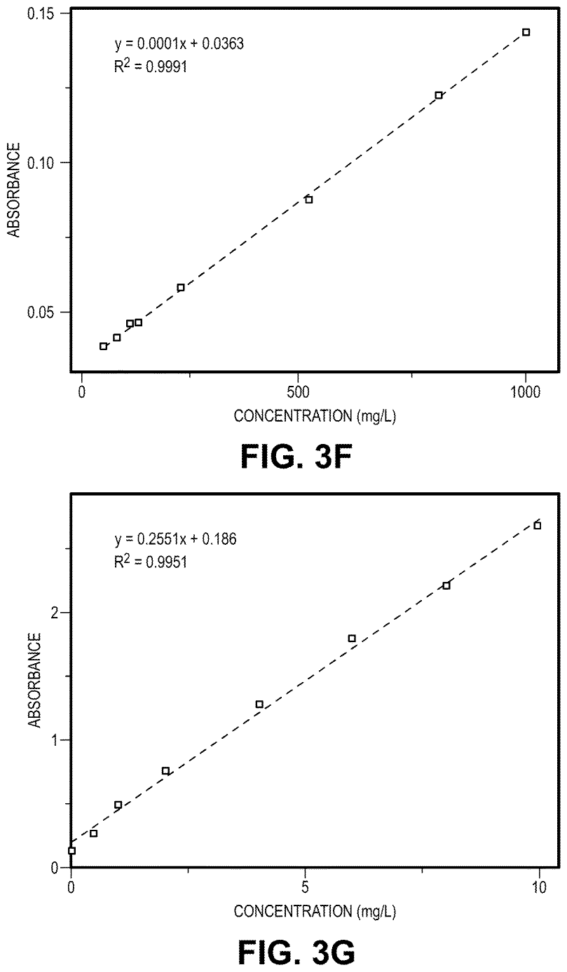

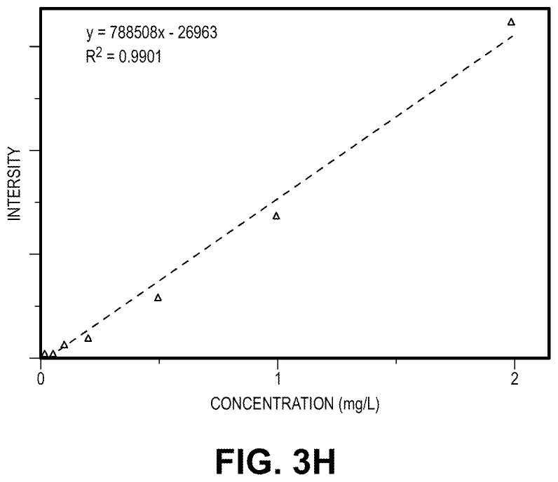

The diffusion and penetration of proteins through the framework membrane and the covalent binding between proteins and the substrate during the diffusion process could be visualized by confocal imaging. For sample preparation, NHS@MF membranes were immersed into 50 mg/L Ab-HIS-647 for 30 min and were washed three times using the PBS buffer before measurements. A laser scanning confocal microscope (FV 1000 system, Olympus America) was utilized. Using a 60× bright field objective and 647 nm (Ar laser) excitation, 665-755 nm emission was collected for the Alexa Fluor™ 647 conjugate used in this experiment. The images were acquired at 640×640 pixels with 12.5 s/pixel scanning speed. As shown in FIG. 3A, Ab-HIS-647 could completely penetrate through inside the NHS@MF membrane and homogeneously distribute and become immobilized on the skeleton of the NHS@MF membrane. With the video S3 in support information (scanning from the bottom to the top of the membrane), it is clear that the immobilization of the protein is uniformly homogeneous in the different layers vertically.

The amount of NHS immobilized on NHS@MF was measured and compared to a nanofibrous membrane with the same area and treat (5% DSC in 100 mL 1,4-dioxane at 70° C.). Per a unit square meter, the NHS@MF showed a higher capacity of NHS than the nanofibrous membrane (NHS@NF) (FIG. 3B). Structurally speaking, even though the nanofibers have higher specific surface area than that of the MF, they are electrospun into nanofibrous membranes with layers of microporous webs. The microsized pores of the layered webs could be further reduced vertically after randomly overlaying together forming NF membranes in varied thickness. The effective pore sizes of the NF membranes could be reduced to one of hundreds or thousands of the measured pore sizes in the vertical direction, which is perfect for serving as a filter [6]. The significantly reduced pore sizes of NF membranes could prevent diffusion of large biomolecules, such HIgG, leading to reduced loading amount shown in FIG. 3B.

Different from NF membrane materials, the MF framework structure can allow free diffusion and penetration of large molecules through the membranes. When the NHS-MF membranes were employed in immobilization of HIgG in varied concentrations (5 mg/L, 1 mg/L, 0.5 mg/L), as shown in FIG. 3C, the amounts of the antibody used and immobilized on MF were corelating well, indicating that the large biomolecules are homogeneously distributed in the MF membrane. Such a structural feature is quite unique for samples with varied volumes, especially in applications of detecting trace amounts of targets in fluid systems that are below normal detection limits since large volume could increase bindings of targets with the immobilized sites.

In a direct visualization comparison between MF and NHS@MF in immobilization of antibodies was conducted by adding 100 μL 0.5 mg/L Ab-HIS-HRP solution to these two membranes in same size and thickness, respectively. Subsequent coloration step of addition of hydrogen peroxide and TMB dye resulted in blue color in varied intensities. FIG. 3D shows that after thoroughly washing, both NHS-treated membranes showed clear color signals than pristine membranes (insert in FIG. 3D) and NHS@MF revealed much higher intensity, proving the DSC modification on both materials and MF structural features.

### 3.3 Direct ELISA on the Melamine Foam Membranes

To demonstrate applicability of the MF in biosensing, direct ELISA was employed on the NHS@MF membranes. An SP-RBD with different concentrations from 0 to 100 mg/L were immobilized on the NHS@MF membranes, and an Ab-HIS-HRP was introduced to specifically bind with the immobilized protein and generate colorimetric signals from the interaction between the HRP and a TMB substrate (Scheme. 1(a)). To find a proper concentration of HRP and enzyme-substate reaction time, optimization experiments were conducted. A concentration of the Ab-HIS-HRP at 1 mg/L was identified as the optimal concentration and a the reaction time of 5 min between the TMB substrate and HRP were chosen accordingly. Besides, to demonstrate the specificity of the assay, different control assays were conducted and the results were collected. In addition to the negative control experiments without the use of the HRP, there was no color or very low response in terms of color change in the absence of SP-RBD.

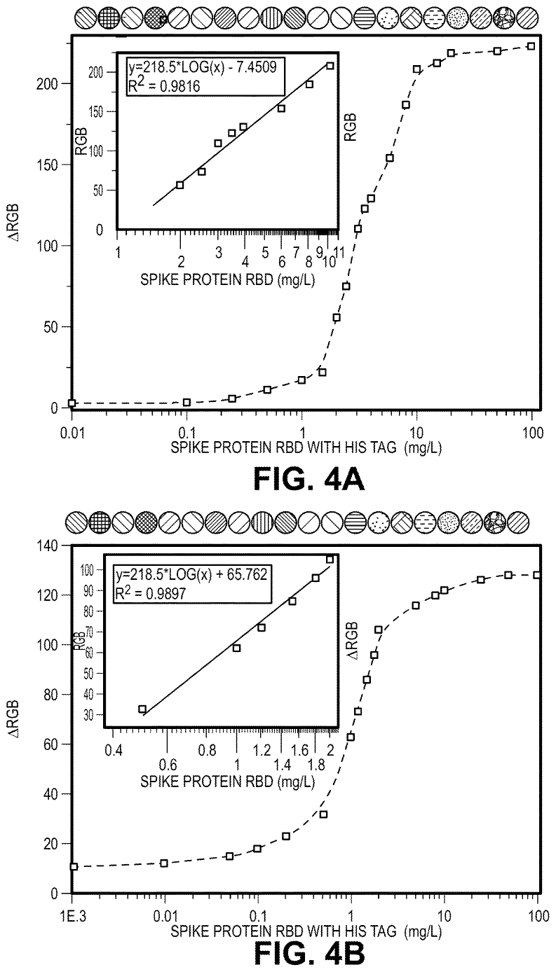

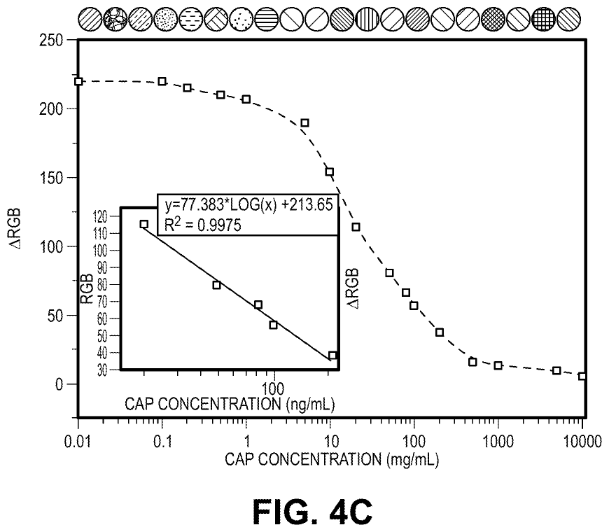

To explore the sensitivity of the biosensing material in detecting target agents, a detection assay with the use of various SP-RBD concentrations (0 to 100 mg/L) was conducted, and the naked-eye readable blue color signals corresponding to different concentrations of the SP-RBD are shown in FIG. 4A. By examining the color intensities via the photoshop software following the equation of ΔRGB=RGBbackground−RGBmembranes, where RGBbackground is the R value of the white background (no HRP), and RGBmembranes is R value of the NHS@MF membranes, the linear equation for the colorimetric assay was fitted to be y=13.67x+2.48 (R²=0.97) between the protein concentrations of 0.1 mg/L to 1.5 mg/L. Naked eye recognizable SP-RBD reached at 1 mg/L level with a limit of detection (LOD) at 0.52 mg/L with the help of a smartphone and further analysis from software for the direct ELISA sensor (FIG. 4A).

### 3.4 Sandwich and Competitive ELISA on the Melamine Foam Membranes

With these solid results of direct ELISA, sandwich and competitive ELISA could also be employed on the NHS@MF membranes. As an evidence of showing the MF membrane as a sandwich ELISA sensing material, SP-RBD was also employed as the detecting target with a testing protocol shown in Scheme 1(b). In the presence of the Ab-SP immobilized on the NHS@MF, the SP-RBD with HIS-tag could be recognized by the antibody. Then the introduction of the Ab-HIS-HRP would generate colorimetric signals from the interaction between the HRP and TMB substrate as shown in Scheme 1(b). To minimize the background signal of control groups (without primary antibodies), we optimized the type and concentration of the blocking buffer. The colorimetric signals of the sample groups blocked with skim milk was lower than that of the groups blocked with BSA under the same treatment, and the group blocked with 3% skim milk presented the lowest colorimetric signal compared with other concentrations of skim milk solution. Therefore, we chose skim milk (3%) as the blocking and dilution buffer in the following experiments.

To explore the sensitivity as the sandwich ELISA sensing material, different S-protein concentrations (0 to 100 mg/L) were employed, and the naked-eye readable color signals corresponding to different S-protein concentrations are shown in FIG. 4A. By examining the intensity of colorimetric signals following the same procedure as in the direct ELISA, the linear equation for the colorimetric assay was fitted to be y=101.65x+18.03 (R²=0.99) between 0.01 mg/L to 0.2 mg/L. Naked eye recognizable SP-RBD reached at 0.1 mg/L level with a limit of detection (LOD) at 0.047 mg/L with the help of a smartphone and further analysis from software for a sandwich ELISA sensor.

A competitive ELISA assay was employed for testing the quantitative sensing ability of the MF membranes on chloramphenicol (CAP), which is used in aquaculture products as an antibiotic. The detection procedure is schematically described in Scheme 1(c). Different from the other two assays, an unlabeled antigen from samples and a labeled antigen competes for binding to the immobilized antibody on the MF. A decrease in color signal from the MF membranes indicates the presence of the antigen in samples when compared to control groups with the labeled antigen alone. To explore the sensitivity of the MF in the competitive ELISA sensing, a detection assay at different CAP concentrations (0 to 10000 ng/mL) and corresponding naked-eye readable color intensities are shown in FIG. 4C. By examining the intensity of colorimetric signals following the same procedure employed in both direct and sandwich ELISA assays, a linear equation for the colorimetric assay was fitted to be y=−122.88x+226.13 (R²=0.96) between 0.01 ng/mL to 0.20 ng/mL of CAP. Naked eye recognizable CAP concentrations reached at 1 ng/mL level with a limit of detection (LOD) at 0.096 ng/mL with the help of a smartphone and further analysis from the software for a competitive ELISA sensor.

### 3.5 Impact of Sample Volumes in Immunoassays

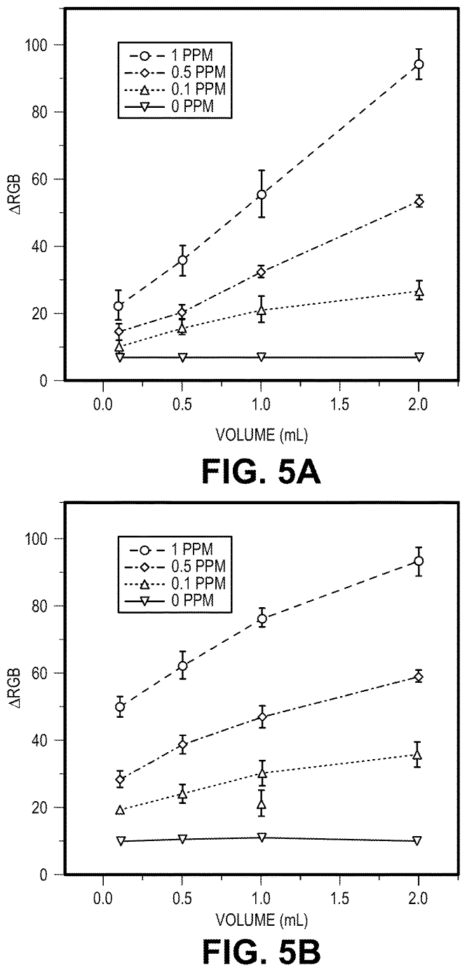

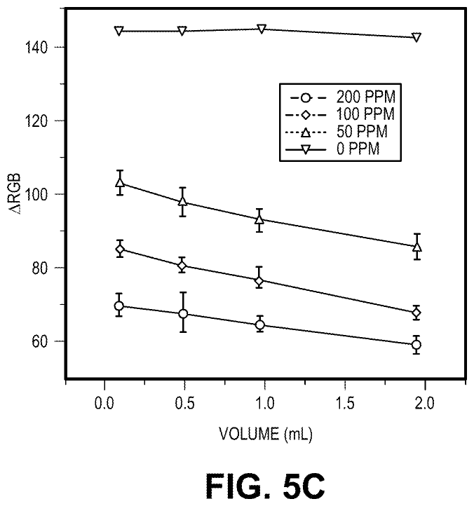

Traditional ELISA assays use a very narrow range of sample volumes. Recently, p-ELISA managed to further scale down the volume through miniaturization of sample sizes. However, different from point-of-care clinical analysis, pollutants could be in very low concentrations in environment and treated industrial wastes that have abundant amounts of samples. Increasing the test sample volume could magnify the intensity of signals to improve the sensitivity of the detection. In the measurement of diffusion behavior of large biomolecules, MF membranes revealed volume responsive color signals as shown in FIG. 3C. The framework structure of the FM allows fluids run through the membranes rapidly without much resistance, even with increased thickness (FIG. 1A). The impact of varied sample volumes on the MF sensing applications in three immunoassays was investigated, the varied volumes of target samples (100 μL, 500 μL, 1 mL, 2 mL) were applied in each experiment. Here, except for the step of analyte addition, the testing steps followed the same protocols as we mentioned above. As shown in FIG. 5A, in a direct ELISA sensing test, by changing the sample volume from 100 μL to 2 mL, the color intensities of the MF sensing material changed dramatically and almost linearly under varied target concentrations of 0.1 ppm, 0.5 ppm, and 1 ppm for S-protein, respectively. Higher target concentration resulted in much stronger signal intensity, while for the very low concentration (0.1 ppm) of S-protein, the intensity was increased coordinately with the increase of the sample volume (FIG. 5A). Similar results were observed on the sandwich assay tests of the S protein (FIG. 5B): In competitive ELISA, the units of intensity inversely changed corresponding to increased concentrations of CAP from 50 ng/mL, 100 ng/mL, to 200 ng/mL, respectively (FIG. 5C). In all three types of ELISA sensing tests, increasing volume of samples led to profoundly stronger colorimetric signal differences, which could improve the sensitivity and broaden the application range. Such a unique feature has not been observed from other sensing materials.

Scheme 1 shows the mechanism of NHS@MF based (FIG. 2L) direct ELISA, (FIG. 2M) sandwich ELISA, and (FIG. 2N) competitive ELISA. The figure key is hsown in FIG. 2O.

### 3.6 Simultaneous Detection of Multiple Targets

The structural feature of MF membranes also provide applications of additive sensing of multiple targets simultaneously in one integrated system. As illustrated in FIG. 6A, antibodies of both Ab-CAP and Ab-CPS were immobilized on two different NHS@MF membranes, respectively. After blocking with BSA, these two membranes (5 mm in diameter) were mounted into a syringe needle pocket as a filtering sensing device, and 2 mL of a mixture of CAP and CPS in specific concentrations, together with CAP-HRP and CPS-HRP in a concentration of 1 mg/L each was filled into a 20 mL syringe and flow through the filtering needle with a flow rate of 6 mL/h controlled by a SyringeONE programmable syringe pump (NewEra Instruments. USA) (FIG. 6B). As shown in FIG. 6C, nine groups of the mixtures were tested and collected through the sensing device following the varied concentrations of CAP and CPS in the FIG. 6C. The intensity of the colorimetric signals of the first-layer membranes showed an increasing trend and that of the second-layer membranes showed a decreasing trend, indicating that simultaneous detection of CAP and CPS could be achieved without any interference of the two targets in the same system, an advantage of potential additive detection of multiple targets in one system.

### 3.7 Comparison of Immobilized Antibodies on MF, NF, and NP.

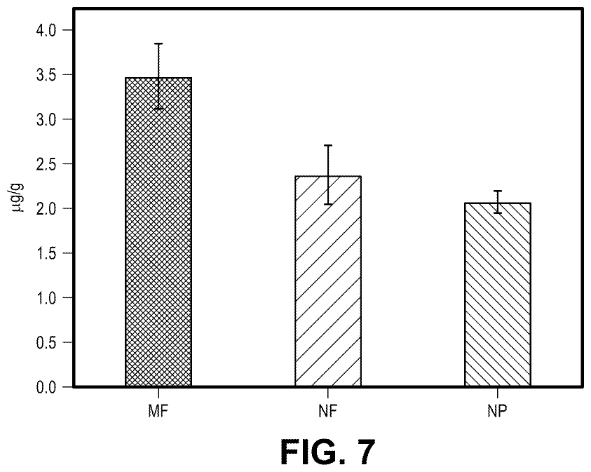

FIG. 7 shows the immobilized antibody amounts on NHS@MF, NHS@NF, and nitrocellulose paper (NP) from 1 mg/L of 100 μL of antibody solution.

### 3.8 Optimization Experiments for Direct ELISA Sensor.

100 μL of the SP-RBD-His in a concentration of 5 mg/L were added to the NHS@MF membranes and incubated for 30 min under gentle agitation; the membranes of the control groups were immersed in PBS during the incubation. Then the membranes were exposed to 3% BSA (200 μL) to block the remaining active sites. Subsequently, 100 μL of Ab-HIS-HRP with different concentrations ranging from 0.1 mg/L to 2 mg/L were added to the membranes, respectively. After 20 min, the membranes were first washed with tween-20 (0.05%) and then washed with PBS buffer and dried in air. 25 μL of TMB substrate (ThermoFisher) was then applied onto the membranes, and membranes were placed in an LED lightbox (E mart). The colorimetric signal from the interaction between HRP and TMB substrate was captured by a smartphone (iPhone 8) and analyzed using Photoshop (Adobe) software. The difference of ΔRGB values between the sample (2 mg/L SP-RBD-His) and control (no SP-RBD-His) groups with the treatment of varied concentrations of Ab-HIS-HRP were recorded in the Table below along with different reaction times between the TMB substrate and HRP. The table below is data from the optimization tests for HRP concentrations and reaction times between HRP and TMB substrate.

| | Concentration | | | | | |
|---|---|---|---|---|---|---|
| Time | 2 | 1 | 0.75 | 0.5 | 0.25 | 0.1 |
| (min) | mg/L | mg/L | mg/L | mg/L | mg/L | mg/L |
| 1 | 152.04 | 141.36 | 93.49 | 92.61 | 60.97 | 28.12 |
| 5 | 122.94 | 181 13 | 158.99 | 154.24 | 142.09 | 119.11 |
| 10 | 109.14 | 132.33 | 142.11 | 144.65 | 150.67 | 150.65 |
| 15 | 78.94 | 99.69 | 129.48 | 136.86 | 145.66 | 149.88 |
| 20 | 69.01 | 88.44 | 119.46 | 130.35 | 147.13 | 154.04 |

### 3.9 Specificity of the Assay

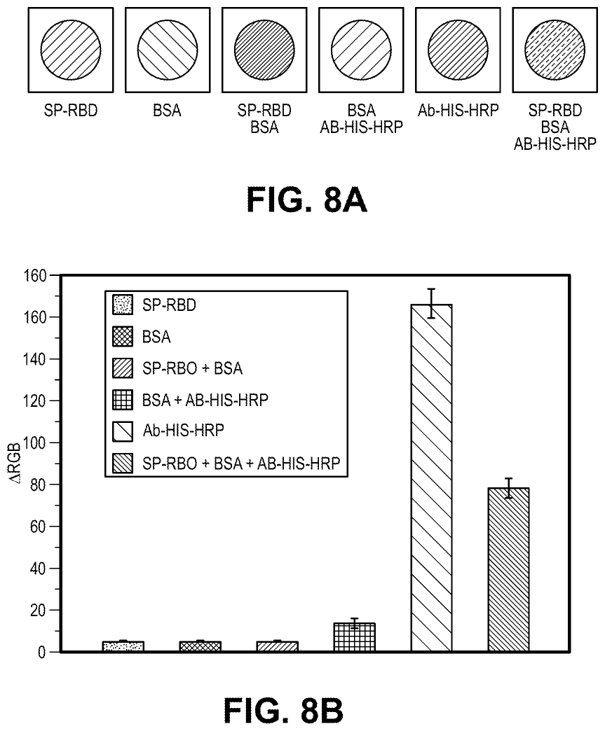

FIG. 8 shows the specificity of the assay. FIG. 8A shows images of the NHS@MF membranes with different treatments after the addition of TMB substrate: 100 μL SP-RBD-His (2 mg/L): 200 μL BSA (3%); 100 μL SP-RBD-His (2 mg/L), and then 200 μL BSA (3%): 200 μL BSA (3%) and then 100 μL Ab-HIS-HRP (1 mg/L); 100 μL Ab-HIS-HRP (1 mg/L); 100 μL SP-RBD-His (2 mg/L), 200 μL BSA (3%), and then 100 μL Ab-HIS-HRP (1 mg/L). FIG. 8B is a bar diagram for the ΔRGB observed from the images.

### 4.0 Optimization of the Type and Concentration of the Blocking Buffer

The NHS@MF membranes were exposed to 1%, 3%, and 5% BSA solution and 1%. 3%, and 5% skim milk solution, respectively. The membranes were then immersed into 10 mg/L SP-RBD-His for 20 min and then exposed to 1 mg/L Ab-HIS-HRP under gentle agitation for 20 min subsequently. The results were then collected after several times washing with the PBS buffer.

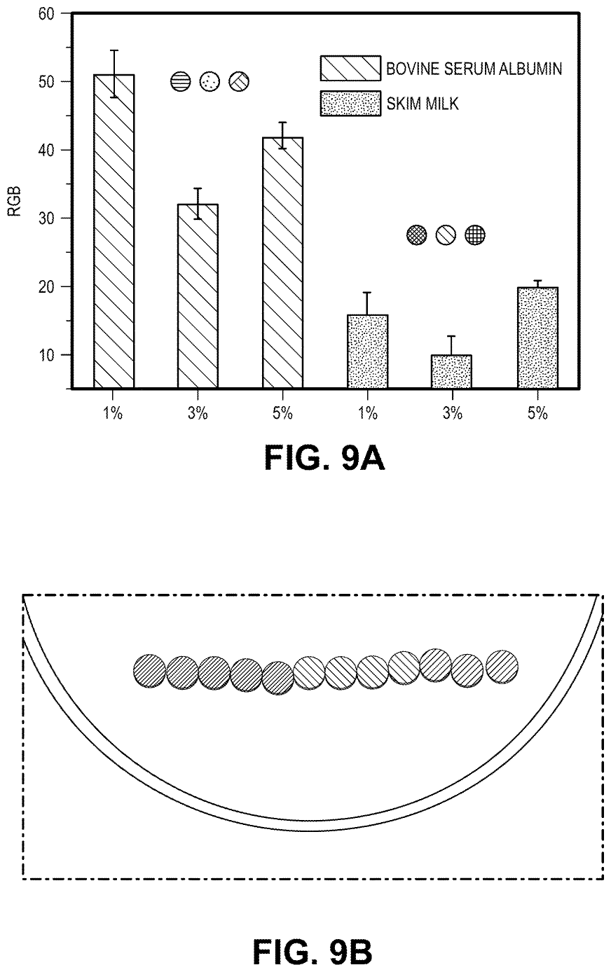

FIG. 9A shows blocking optimization. Optical images and the intensity of colorimetric signals of 1%, 3%, 5% BSA treated membranes and 1%, 3%, 5% skim milk treated membranes. FIG. 9B shows optical images of a sensing test of NHS@MF after the material was exposed to different concentrations of CAP. (without image crop).

### Conclusions

Unique rapid, sensitive, and additive and volume responsive colorimetric biosensing materials were fabricated from using chemically modified reticulated melamine foam materials and can be applied in competitive, direct, and sandwich ELISA biosensors. The sensing materials demonstrated promising detection sensitivity to a SARS-CoV-2 spike protein, a transmembrane protein of SARS-CoV-2 virus, and chloramphenicol (CAP), often used as an antibiotic. Naked eye recognizable SARS-CoV-2 spike protein reached at 1 mg/L level with a limit of detection (LOD) at 0.5 mg/L with the help of a smartphone and further analysis of RGB value from an APP software for a direct ELISA sensor, and at 0.1 mg/L and a LOD of 0.05 mg/L from using the smart phone program for a sandwich ELISA sensor. In addition, using a competitive ELISA, chloramphenicol (CAP) can be detected at 1 ng/mL level with the naked eye and at 0.1 ng/mL with the help of a smartphone. Moreover, due to the excellent mechanical properties and framework structure of the MF, diffusion of the analyte through the different membrane layers is fast and homogeneous in all directions and suitable for detection of large volume of samples in low concentration of targets and multiple targets simultaneously in one integrated system with minimal interference from targets. The successful fabrication of such biosensors significantly improves the sensitivity and broaden the applications of biosensing materials.

### Example 2 Shows that the Foam Based ELISA can be Used to Detect Foodborne Pathogens

### 1—Introduction

Foodborne illnesses represent significant public health challenges worldwide¹⁸. Among these. E. coli O157:H7 is a particularly concerning pathogen due to its low infectious dose and severe health consequences^{19,20}. This specific serotype of E. coli can cause diseases ranging from mild diarrheal illness to severe conditions like hemorrhagic colitis and hemolytic uremic syndrome, which can lead to kidney failure or death in extreme cases. E. coli O157:H7 causes an estimated 63,000 hemorrhagic colitis cases annually in the United State^{21,22}. Its low infectious dose, high pathogenicity, and a potential risk of contamination in water and food sources make it a significant threat to food safety and public health^{23,24}. Currently, the detection of E. coli O157:H7 in food and water samples has relied heavily on conventional methods including culture-based assays, polymerase chain reaction (PCR), and isothermal amplification^{25,26,27,28}. While these methods have proven effective over time, they possess several limitations. The culture-based assay, with its high reliability and sensitivity, is considered the gold standard in the field of bacterial detection²⁹. However, it is time-intensive (2-3 days) and requires highly trained personnel, making it unsuitable for rapid onsite detection³⁰. PCR's exceptional sensitivity is counterbalanced by its need for expensive equipment and complex preparation procedures^{31,32}. Isothermal amplification methods amplify DNA at a consistent temperature, contrasting the temperature cycling of PCR. While adept at detecting pathogens in trace amounts, this method can be hindered by complex primer design and contamination risks, potentially leading to a high false-positive rate³³. Other methods, including flow cytometry, gas chromatography. Fourier transform infrared spectroscopy (FTIR), and Raman spectroscopy, etc., also require professional tools, being both costly and time-consuming^{34,35}. The existing diagnostic methods prove challenging to implement in low-income countries, where high mortality rates prevail due to a lack of adequate diagnostic tools^{36,37}. Therefore, there has been a pressing need for a more efficient, affordable, and rapid detection method to combat this public health threat.

Currently, paper-based colorimetric biosensors have attracted a great deal of attention in detecting pathogenic bacteria in food and water with naked eyes³⁸. The straightforward design and operation of paper-based ELISA (p-ELISA) colorimetric sensing systems make them an appealing choice for disposable on-site detection systems, which could potentially be used by untrained personnel^{39,40,41}. However, a variety of technical challenges restrict their use for assessing food safety in terms of microbial contamination. Detecting a small number of pathogenic bacteria within a large volume of a food or water sample proves difficult for these conventional systems due to their relatively low detection sensitivity. Additionally, the complexity of food matrices—including fats, proteins, saccharides, fibers, and various salts—can significantly interfere with the separation of target bacteria from the food or water sample and the subsequent color development reaction. A key factor contributing to the limitations of p-ELISA is the heterogeneous structures of the papers and fibrous membranes used in the process, especially along the vertical direction, inhibiting penetration of large biomolecules through the membranes, resulting in lower than the expected number of biomolecules incorporated onto surfaces of fibers inside the media⁴². Even though the media is often described as having a three-dimensional structure, the inner part of the materials is seldom fully utilized. Particularly when whole cells of microorganisms are used as antigens, their micrometer sizes restrict them from diffusing and penetrating into and even if they manage to traverse the layered narrow porous structure, they often become trapped and are difficult to wash off through the system⁴³. Such a structural feature could consequently lower the sensitivity of p-ELISA sensors made by nitrocellulose membranes, filter papers, and even nanofibrous membranes, resulting in inhomogeneous colorimetric signals, strong sample matrix effects, and high false-positive rates of p-ELISA sensors⁴⁴. Hence, an ideal media with a three-dimensional, homogenous, and open macroporous structure that permits the free migration of large biomolecules in all directions is envisioned as a better material for such ELISA biosensors for bacteria cells.

In our previous studies, we demonstrated that the foam-based ELISA (f-ELISA) using melamine foam (MF) as a medium offers unique advantages⁴⁵. It was proven to be rapid, sensitive, additive, and volume-responsive across different types of approaches, including direct, competitive, and sandwich ELISA by detecting SARS-CoV-2 spike protein and chloramphenicol (CAP). In this context, we believe that f-ELISA is even better suited for E. coli O157:H7 detection, given the larger size of bacteria compared to chemical compounds and proteins. The application of the f-ELISA in detection of bacteria cells could fully demonstrate the advantages of the macroporous features offered by the chemically modified melamine foam. In contrast to conventional ELISA (c-ELISA), which is restricted by the limited surface area of a 96-well plate, and other p-ELISA methods, bacteria as antigens can move freely in every direction within this macroporous 3D matrix. This enhanced freedom of movement facilitates an amplified interaction between the immobilized antibodies and antigens, leading to substantial enrichment and heightened sensitivity in colorimetric detection. The testing process needs less than 1.5 h to complete both preparation and detection, and the results revealed that the sensors made of the MF materials could detect E. coli O157:H7 at 10 CFU/mL level by naked eyes with a limit of detection (LOD) at 5 CFU/mL when supplemented by a smartphone. Following a brief enrichment period of 1 hour, the sensitivity was further amplified to 2 CFU/mL. Interestingly, the sensitivity increases as the volume of the sample increases, making this sensing material highly suitable for testing large-volume fluid samples, such as milk, drinking fluids, agricultural water, etc. In essence, this study paves the way for a rapid, sensitive, and volume-flexible biosensing platform, using E. coli O157:H7 as a proof of concept, which holds promise for the rapid and ultra-sensitive detection of various pathogenic bacteria in real-world applications.

### 2. Experimental Section

### 2.1 Materials

N, N′-disuccinimidyl carbonate (DSC), triethylamine (TEA), 1,4-dioxane, acetone, phosphate-buffered saline (PBS), and 96-well plates were purchased from ThermoFisher Scientific. Escherichia coli 0157 mouse anti-E. coli monoclonal antibody and E. coli rabbit anti-E. coli polyclonal (HRP) antibody were purchased from Lifespan Biosciences (Shirley, MA, USA). Melamine foams were purchased from Swisstek (Brewster, NY, USA). Maximum recovery diluent (MRD) was purchased from Sigma-Aldrich (Louis, MO, USA). Phosphate buffer solution (PBS), tryptic soy broth (TSB), and tryptic soy agar (TSA) were purchased from Fisher Scientific (Fair Lawn, NJ, USA). All other chemicals were of analytical grade and were supplied by Merck (Darmstadt. Germany). Rifampin-resistant E. coli O157:H7 (ATCC700728), E. coli BL21 (ATCC BAA-1025), Listeria innocua (ATCC 33,090) were obtained from ATCC (Manassas, VA, USA). MacConkey agar was supplied from Difco (Sparks, MD, USA). SYBR Green I nucleic acid stain (10×concentrate) was purchased from Invitrogen (Carlsbad. CA, USA).

Morphologies of all MF based samples were analyzed using a scanning electron microscope (Quattro ESEM, Thermo Scientific). An electronic micrometer thickness gauge (Neoteck) was used to measure the thickness of the MF membranes.

### 2.2 Bacterial Culture and Sample Preparation

E. coli O157:H7 was stored in TSB containing 15% (vol/vol) glycerol at −80° C. Prior to the experiments, the glycerol stock was streaked onto tryptic soy agar (TSA) plates and incubated overnight at 37° C. The culture plates could be stored at 4° C. for approximately 30 days. Overnight culture of E. coli O157:H7 was prepared by inoculation of a loopful of culture from the TSA culture plates in 10 mL of sterile TSB and incubation at 37° C. with 200 rpm constant shaking. After an incubation time of 16 h, the E. coli overnight culture was enumerated to have a titer of 10⁹ CFU/mL. An overnight culture of E. coli O157:H7 was centrifuged at 13,000 rpm for 1 min to recover the bacterial cells. TSB was discarded and the cells were washed twice and resuspended with sterile PBS. The E. coli suspension (10⁹ CFU/mL) was diluted in PBS to obtain the different bacterial concentrations.

### 2.3 Measurement of Diffusion of Biomolecules and Bacteria

The diffusion of E. coli O157:H7 within melamine foam (MF) of varying thicknesses, nitrocellulose paper (NP), and nanofibrous membrane (NF) were measured using a side-by-side diffusion chamber (PermeGear Co.). This apparatus consists of two 3.4 mL chambers (a donor chamber and a receptor chamber) connected by a 9 mm orifice. MF membranes at different thicknesses (1-3 mm), as well as NP and NF, were separately placed between the two chambers. The chambers were tightly sealed and situated in a water bath to maintain a consistent temperature of 25° C. To pre-wet the membranes, each chamber was filled with 3 mL of a PBS solution for 15 minutes. Following this, 3 mL of E. coli O157:H7 suspension (10⁷ CFU/mL) was injected into the donor chamber. Stirring bars were set in both chambers, operating at a speed of 750 rpm throughout the tests. At regular time intervals, 1 mL of the sample solution was extracted from each chamber and added back to the chambers after the measurement via Ultraviolet-visible spectroscopy (UV-Vis) at the wavelength of 600 nm⁴⁶. The concentration of E. coli O157:H7 was determined with the UV-Vis (Thermo Scientific), based on calibration curves provided in the supporting information (FIG. 1a). The subsequent analysis of protein or bacteria concentration in the receptor chamber over increasing time intervals allowed for an assessment of the diffusion properties of the biomolecules through the MF membranes.

### 2.4 Vertical Flow Test Through Materials

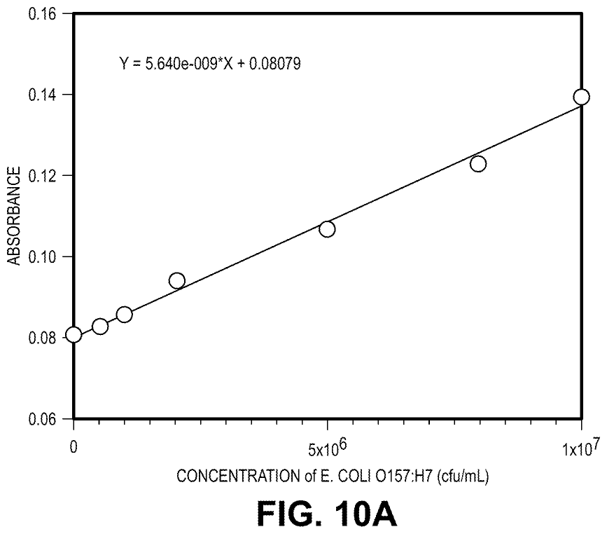

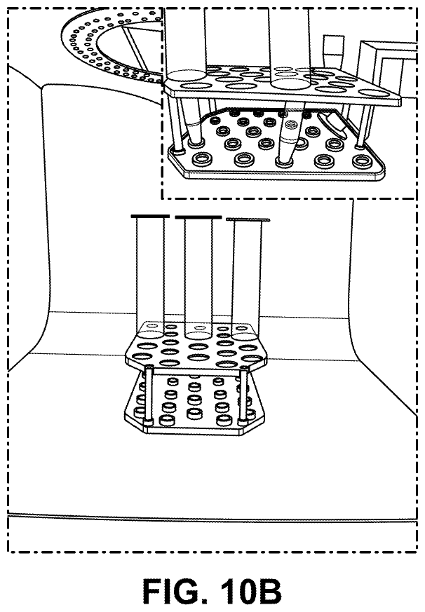

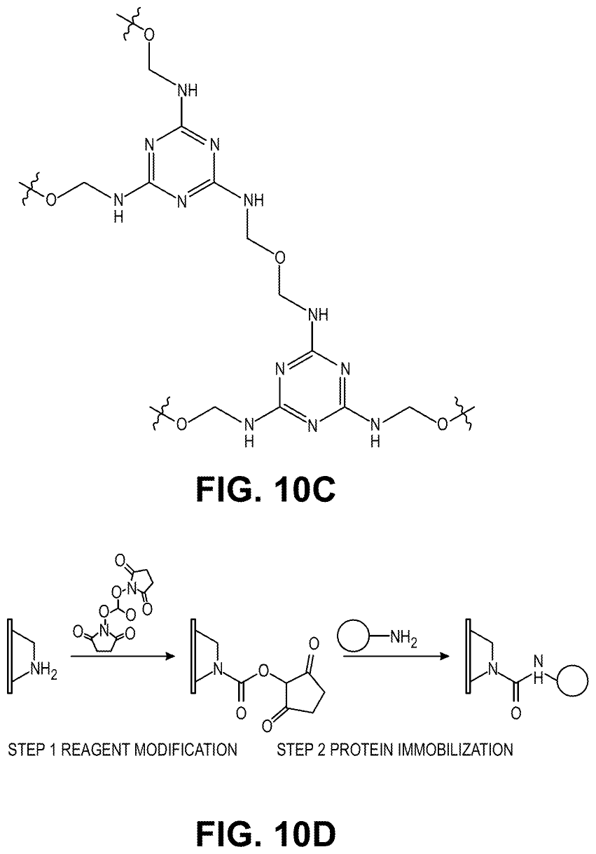

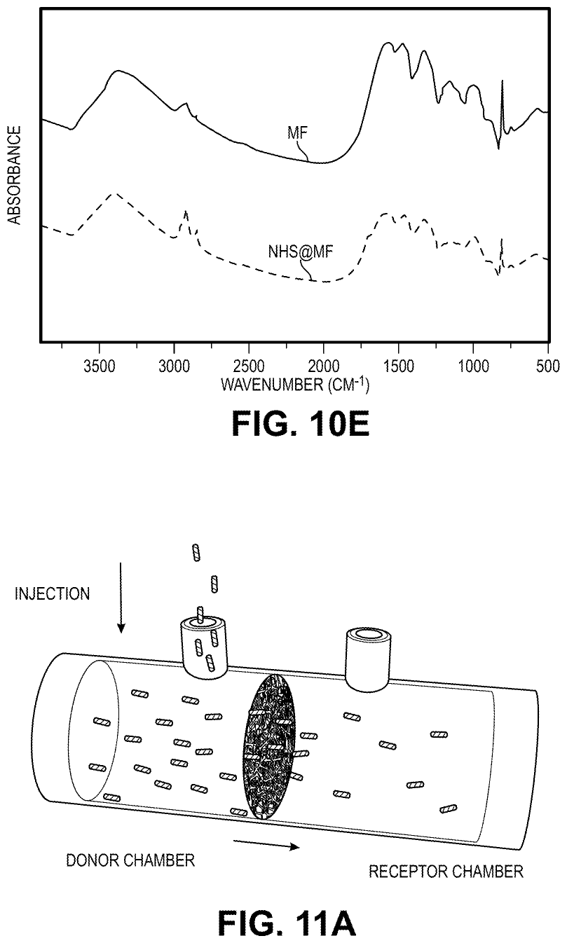

The vertical flow test was carried out by separately placing discs of MF at different thicknesses (1-3 mm), NP and NF in the bottom of a 20-mL syringe creating a filtration column (FIG. 10b). A 1-mL of E. coli O157:H7 suspension at concentration of 10³ CFU/mL was passed through the columns containing the different testing matrices. The collected filtrates were performed by serial dilution and plate counting using TSA containing 0.05 g/L rifampicin. After passing 10 mL sterile PBS to replace any remaining bacterial solutions, the different discs were transferred into a 15-mL sterile centrifugal tube containing 1 mL of the releasing buffer (MRD with 0.01% lecithin), allowed to stand for 2 min then vortexed vigorously for 1 min to recover the captured bacterial cells⁴⁷. The quantification of the recovered bacterial cells was performed by serial dilution and plate counting using TSA with 0.05 g/L rifampicin.

### 2.5 Foam-Based ELISA Platform Preparation

A typical chemical structure of melamine foams is shown in FIG. 10c. The chemical modification processes of MF were same as reported in a previous publication (FIG. S4a)²⁸. The MF samples were all in circular form of 1.0 mm thickness and 5.0 mm diameter. The structures of MF and DSC-modified MF membranes (NHS@MF) samples were characterized by FTIR with spectra same as the ones reported in literature (FIG. 10d). Then, a 100 μL of Ab-E. coli solution (10 mg/L) was added to the NHS@MF membranes and incubated for 30 min at the room temperature. After the antibodies immobilization, the remaining active sites were blocked using 200 μL of 3% skim milk (SKM), we defined the material as Ab@NHS@MF.

### 2.6 Foam-Based ELISA in E. coli O157:H7 Detection

The analytical performance of the biosensing platform-based MF (f-ELISA) was evaluated by adding 100 μL of E. coli O157:H7 at varied concentrations (ranging between 0 to 10⁷ CFU/mL) to the f-ELISA membranes, and the incubation lasted for 30 min under gentle agitation. After incubation, any unbound bacteria were removed by washing with PBS buffer. Subsequently, 100 μL of Ab-E. coli-HRP (1 mg/L) was added to each membrane. After 20 min, the membranes were washed with PBST (PBS+tween-20, 0.05% v/v) and then washed with PBS buffer and dried in air. 35 μL of TMB substrate (ThermoFisher) was then added onto the membranes, and the membranes were placed in an LED lightbox (E mart). The colorimetric signal from the interaction between HRP and TMB substrate was captured by a smartphone (iPhone 14 Pro Max) and analyzed using Photoshop (Adobe) software. To capture images of the results, a smartphone was positioned 50 cm above the membranes.

In order to further investigate the impact of the sample volume on E. coli O157:H7 detection, varied volumes of samples (From 100 μL to 10 mL) were applied to each experiment. In this study, except for the addition of varied volumes of analytes, the rest steps followed the same protocols as we mentioned above.

To ensure the long-term efficacy and repeatability of the f-ELISA system, we examined the stability and activity of stored antibodies on the modified MF. The Ab@NHS@MF membranes were treated using 10% sucrose as a stabilizer followed by freeze-drying⁴⁸⁻⁴⁹. They were stored at a consistent temperature of 4° C. and assessed over a period of 90 days. At predetermined time intervals, aliquots were retrieved and utilized in the f-ELISA assay to detect E. coli O157:H7 following the same protocols.

### 2.7 Fluorescence Images of E. coli O157:H7 Cells Captured by Ab@NHS@MF

A 1 mL of overnight culture of E. coli O157:H7 was centrifuged at 13,000 rpm for 1 min to recover the bacterial cells. The broth was discarded and the E. coli cells were washed twice and resuspended with sterile PBS. A 100 μL of 10×SYBR green I was added and incubated in the dark for 5 mins. Afterward, the labeled bacterial cells were recovered, washed with sterile PBS three times to remove the excess SYBR green I dye, and resuspended with sterile PBS. The labeled E. coli suspension was diluted in PBS to obtain the cell concentration of 10⁵ CFU/mL. Then, 100 μL of E. coli O157:H7 (10⁵ CFU/mL) were added to the Ab@NHS@MF membranes, and the incubation lasted for 30 min under gentle agitation. Subsequently, the membranes were washed with PBST and then washed with PBS buffer and dried in air. Fluorescence Microscopy Images of SYBR green I labeled E. coli were acquired using a laser scanning confocal microscope (Olympus FV1000) with a tetramethylrhodamine-isothiocyanate (TRITC ex 541 nm/em 572 nm) filter. The image processing software ImageJ was used to convert the acquired fluorescence image files format from TIFF into JPG⁵⁰.

### 2.8 Detection of E. coli O157:H7 in Real Samples

Irrigation water sample was collected from the Campbell tract at the University of California, Davis, which is the Solano County District agricultural irrigation water (Agwater). Prior to spiking, the sample was autoclaved to remove any background noise created by the Agwater. Then, the autoclaved Agwater sample was spiked with E. coli O157:H7 at concentrations range of 10-10⁴ CFU/mL. Non-spiked autoclaved Agwater sample was employed as a control. For analysis the samples using f-ELISA, the Ab@NHS@MF membrane was mounted into a syringe needle pocket, 5 mL of the prepared sample solution was filled into a 20-mL syringe and flowed through the filtering needle with a flow rate of 10 mL/h, controlled by a SyringeONE programmable syringe pump (NewEra Instruments, USA). Moreover, non-autoclaved Agwater sample was tested without spiking using the f-ELISA and the achieved results were compared with the plate counting assay approach using a selective medium (MacConkey agar plates). The presence of red colonies on the MacConkey agar plates indicates the presence of E. coli O157:H7 in the Agwater sample.

### 2.9 Colorimetric Data Processing

Upon the addition of TMB substrate to the Ab@NHS@MF membranes, they were positioned inside an LED lightbox and images were taken using a smartphone camera. The intensity of the color was represented by the red channel (R) from the RGB values⁵¹. In this context, the variation in the red channel intensity could be illustrated by the ΔRGB value, which was obtained by the RGB value difference between the white background and each membrane, as the equation (1):

$$Δ RGB = {RGB}_{background} - {RGB}_{membranes} \qquad ( 1 )$$

where RGB_{background} is the R value of the white background (no HRP), and RGB_{membranes} is R value of the Ab@NHS@MF membranes.

### 2.10 Statistic Analysis

All experiments were repeated three times. Data are expressed as mean±standard deviations (SD). Intergroup comparison was analyzed by Student's t-test (two-tailed). The level of significance was defined as *P<0.05, **P<0.01, ***P<0.001.

The correlation coefficient (R) was used to measure the linear correlation between observed and predicted values. A value of P<0.05 was considered statistically significant. All statistical analyses were performed using GraphPad Prism 8.0.2.

### 3. Results and Discussion

### 3.1 Filtering and Diffusion Test of Bacteria in MF Membranes

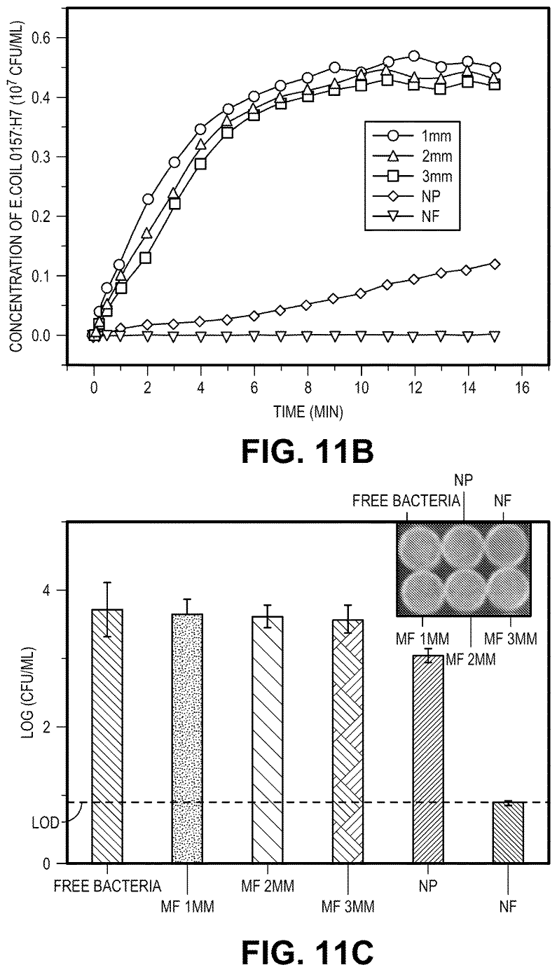

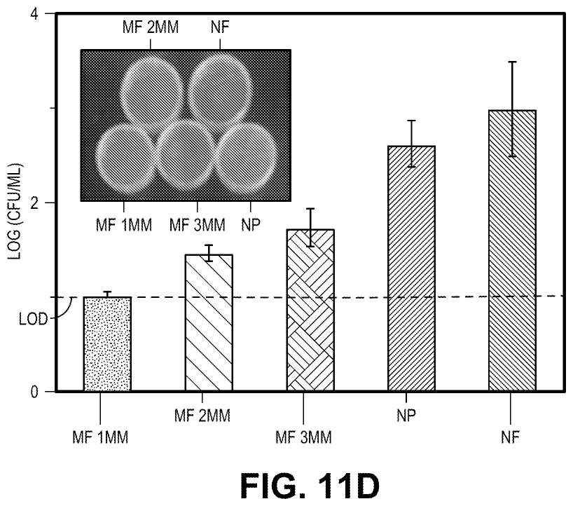

Diffusion of large molecules and particles in chartaceous materials was proven heterogeneous and very slow in vertical directions due to the fact of layered fiber mats and significantly reduced effective pore sizes²⁴. However, the framework of MF materials possesses a unique 3D macroporous fibrous structure and could allow large biomolecules or particles to penetrate through them without much resistance. Based on our previous studies²⁶, we found that compared to the diffusion performances of the biomolecules in various sizes (40 KDa to 150 KDa) in nanofibrous membranes and nitrocellulose papers, which required hours to reach the steady state of diffusion, the thicknesses of MF membranes and sizes of proteins did not show any significant impact and can be ignored if the time of interaction between the MF and substrates is longer than 10 min. However, the diffusion properties of bacteria in the macroporous MF materials could be different, as bacteria cells are in significantly larger dimensions than proteins and other biomolecules—with lengths ranging from 1 to 10 microns and widths between 0.2 to 1 micron⁵². Thus, as depicted in FIG. 11a, a side-by-side diffusion chamber was utilized to investigate the diffusion behaviors of E. coli O157:H7, aligning with our focus on this bacterial strain in subsequent experiments using the innovative f-ELISA system, The concentration changes of E. coli O157:H7 in the receiver chamber versus diffusion times of the cells through MF membranes in varied thicknesses of 1 mm to 3 mm, nanofibrous membrane, and nitrocellulose paper were measured and are plotted in FIG. 11b. In both nanofibrous membranes [poly(vinyl alcohol-co-ethylene), PVA-co-PE] and nitrocellulose papers, the diffusion of E. coli O157:H7 required several hours to attain a steady state. Conversely, in all MF membranes, regardless of their thickness, the steady state of diffusion was achieved in less than 11 minutes. The variation in concentrations observed in the MF membranes with different thicknesses could be attributed to the fact that the increased thickness in MF corresponds to a greater material volume. This increased volume can potentially retain more of the bacterial solution, resulting in a slightly reduced concentration in the Receptor Chamber upon reaching steady state. The diffusion test simulates the process of the MF membranes encountering with bacteria solution samples under a specific stirring rate. As evidenced by the results, the open framework structure, high porosity, and large pore size of the MF allow whole cells to penetrate through the membranes without significant mass transfer resistance. This facilitates the thorough exposure of the MF's 3D framework to pathogens in liquid form, substantially augmenting the likelihood of interactions between the immobilized antibody and E. coli O157:H7, increasing the sensitivity in pathogen detection of the f-ELISA media. A liquid filtering test was also conducted to evaluate the potential application of the MF media in additive filtering sensing devices (FIG. 10b). As shown in FIG. 11c, minimal amount of E. coli O157:H7 was trapped when the solution flew through a 1 mm thick MF disc, and only slight increases in trapped bacteria cells were observed for the 2 mm and 3 mm thick MF discs. Both NF and NP could trap or block more bacterial cells with significantly reduced concentrations of the bacteria shown in the filtered solutions. The non-specific adsorption results of each medium after buffer wash were consistent with the vertical flow test (FIG. 11d). No bacteria were non-specifically bound on the 1 mm thick MF disc. In contrast, NP and NF samples exhibited significant retention of the bacteria. This retention could elevate the background in biosensors using these two materials as detection platforms, potentially increasing the false-positive rate, and reducing the sensitivity of the assay. Therefore, the 1 mm thick MF was selected for subsequent tests to optimize the accuracy and sensitivity of the f-ELISA for E. coli O157:H7 detection.

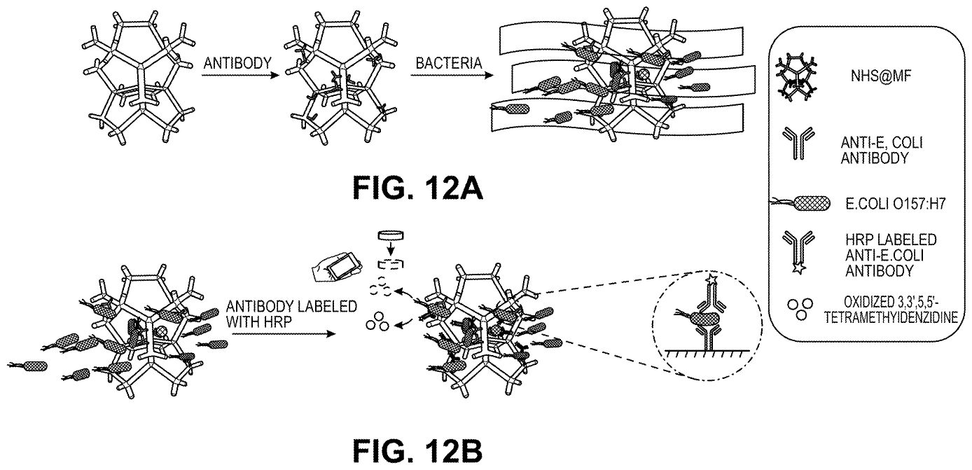

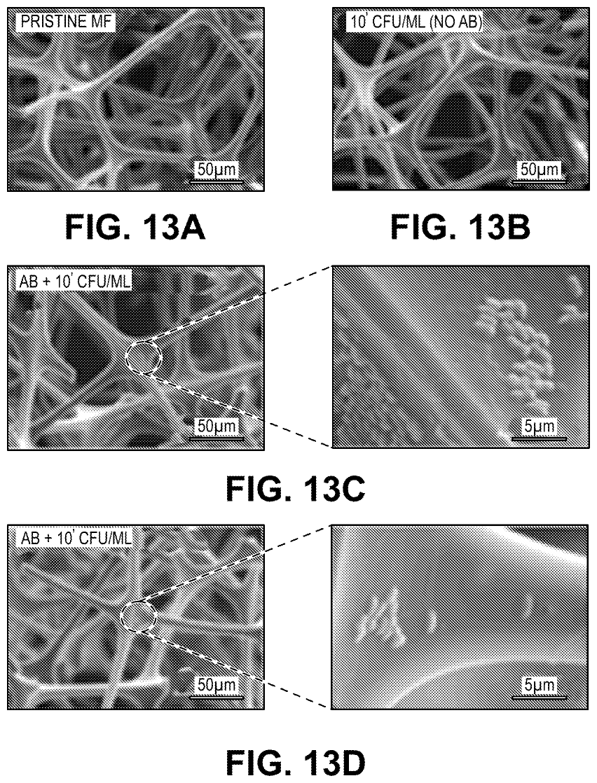

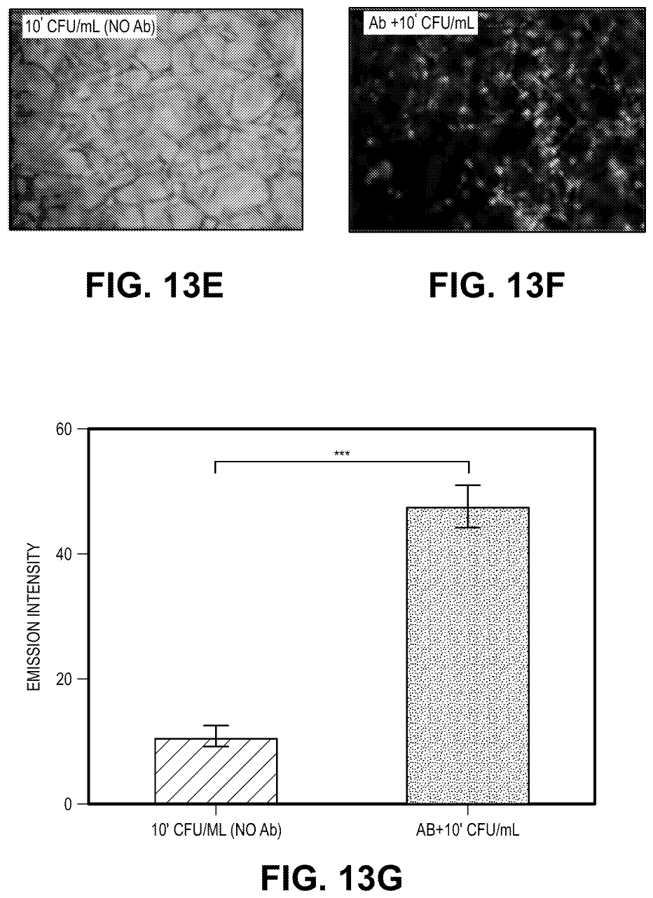

As our previous results indicated, to covalently immobilize proteins on the MF, chemical modification of the secondary amino groups on the material is necessary (FIG. 10c), which is achieved by using DSC to introduce the N-hydroxysuccinimide (NHS) functional groups on the material (NHS@MF) that can readily react with amino groups in proteins. The reactions of chemical modification and protein immobilization on the MF are shown in FIG. 10d. Fourier-transform infrared spectroscopy (FTIR) proved successful incorporations of the reactive groups (NHS) onto the MF, based on the carbonate peak of NHS@MF at 1730 cm⁻¹⁵³. The loading capacity of antibodies on NHS@MF was higher than both NHS@NF and nitrocellulose paper per mass, generating more reactive sites for target molecules than the regular materials used in the p-ELISA sensors²⁶. After the immobilization of anti-E. coli O157:H7 antibodies, the Ab@NHS@MF should be able to capture the target bacteria specifically from the liquid samples as illustrated in FIG. 12a. From SEM characterization results, it is evident that the morphology of the MF framework structures remains unchanged after chemical modification, protein immobilization, and bacteria capture (FIG. 13a-c). As demonstrated in FIG. 13b, in the absence of immobilized antibodies on the material, no unspecific binding was observed. This suggests a low background in subsequent f-ELISA tests, corroborating the results from the diffusion and vertical flow tests mentioned earlier. Furthermore, as shown in FIG. 13c,13d, it is very clear that when the Ab@NHS@MF membranes were employed in the capture of E. coli O157:H7 in varied concentrations (10³ and 10⁵ CFU/mL), the amounts of the bacteria used and captured on the MF media were related. From the results of the fluorescent microscope, after being modified with anti-E. coli O157:H7 antibodies, blocked with SKM, and incubated with E. coli O157:H7 solution at a concentration of 10⁷ CFU/mL, the difference in signal intensities of NHS@MF and Ab@NHS@MF is statistically significant, and the green dots, representing the bacterial cells, homogeneously distribute through the framework of the entire membrane (FIG. 13 e-g). Given these attributes, f-ELISA could be a highly promising platform for detecting whole-cell antigens.

### 3.3 Analytical Performance of f-ELISA

The f-ELISA sensing system, rooted in its novel approach for detecting whole-cell antigens, showed great potential in the initial experiments. In the following section, we will detail the performance metrics of this innovative system, focusing on its specificity and sensitivity. The general f-ELISA procedure of the detection of E. coli O157:H7 is shown in FIG. 12a. In the presence of the anti-E. coli O157:H7 antibodies immobilized on the NHS@MF, the E. coli O157:H7 could be recognized and then captured by the MF based sensor media. Then the introduction of the Ab-E. coli-HRP into the system would generate colorimetric signals from the reaction between the HRP and TMB substrate. By analyzing the colorimetric signals of the f-ELISA obtained from the pictures taken by a smartphone (FIG. 12b), the detection of bacteria can be achieved on-site.

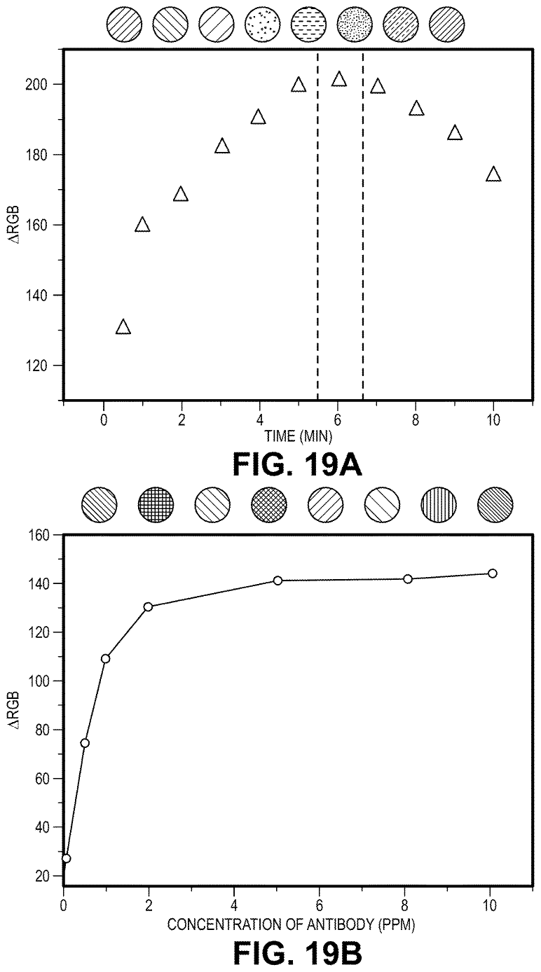

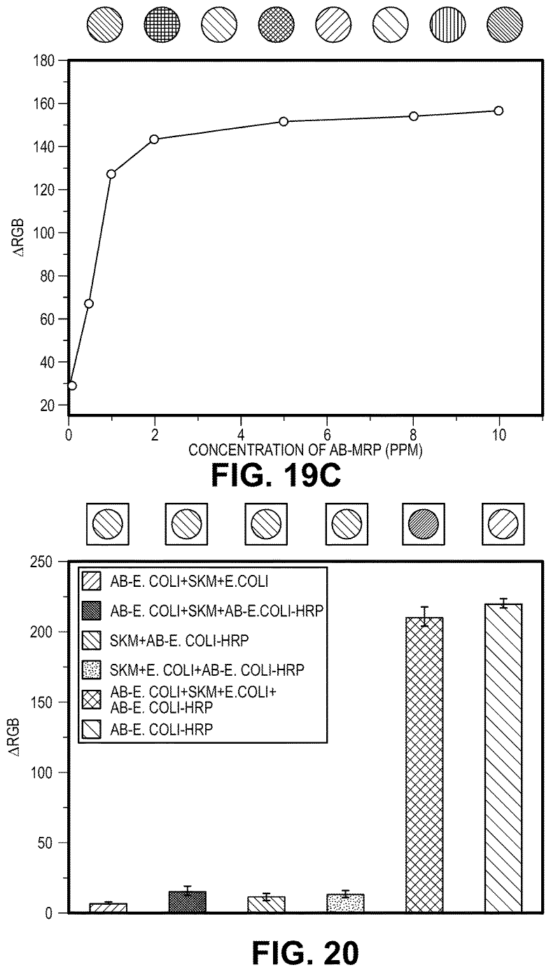

First, the optimization of experimental conditions including the concentrations of antibody and HRP, and enzyme-substate reaction time were conducted with results shown in FIG. 19. The optimal concentrations of Ab-E. coli and Ab-E. coli-HRP were identified as 5 mg/L and 2 mg/L, respectively, and a reaction time of 6 min between the TMB substrate and HRP was chosen accordingly. To attest the specificity of the assay, we carried out an array of control experiments, including the tests without Ab-E. coli-HRP, Ab-E. coli, SKM, or target, respectively. The data from these controls, shown in FIG. 20, further confirmed the robustness of the f-ELISA sensor. In addition to the negative control experiments without the use of the HRP, there was no color or very low response in terms of color change in the absence of Ab-E. coli (FIG. 20), aligning perfectly with the results of SEM and fluorescent microscope (FIG. 13).

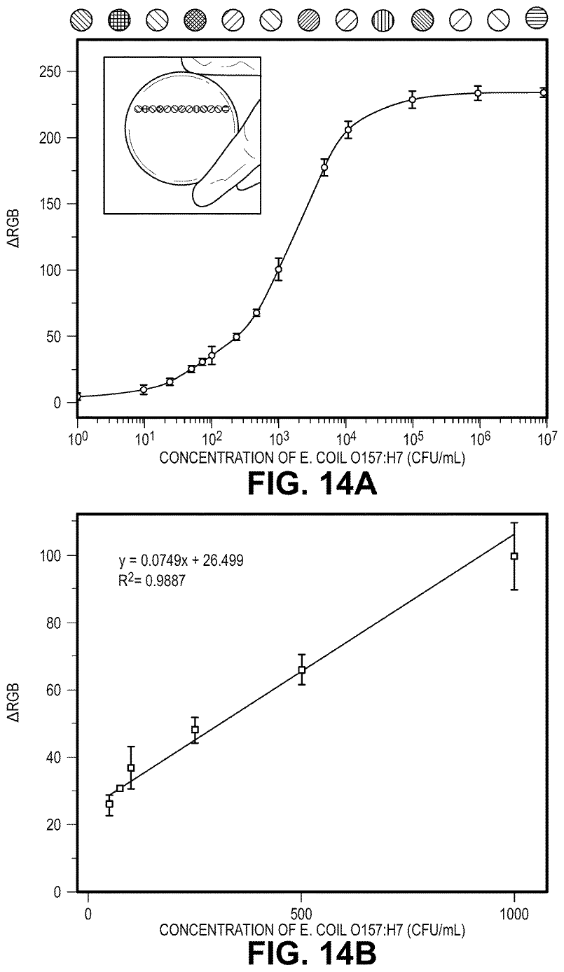

To explore the sensitivity of the f-ELISA in detecting target agents, a detection assay using 200 μL of varied E. coli O157:H7 concentrations (0 to 10⁷ CFU/mL) was conducted, and the naked-eye readable blue color signals corresponding to different concentrations of the E. coli O157:H7 are shown in FIG. 14a. By analysis of the color intensities via the photoshop software using the equation (1), the linear equation for the colorimetric assay was determined as y=0.0749x+26.499 (R²=0.989) between the bacteria concentrations of 50 CFU/mL to 10³ CFU/mL (FIG. 14b). Based on FIG. 14a, the color signal for E. coli O157:H7 at 50 CFU/mL level was naked eye readable, while a limit of detection (LOD) of 10 CFU/mL was achieved using a smartphone acquired image and further analysis of the image from Photoshop for the f-ELISA sensor.

### 3.4 Impact of Sample Volumes in Immunoassays

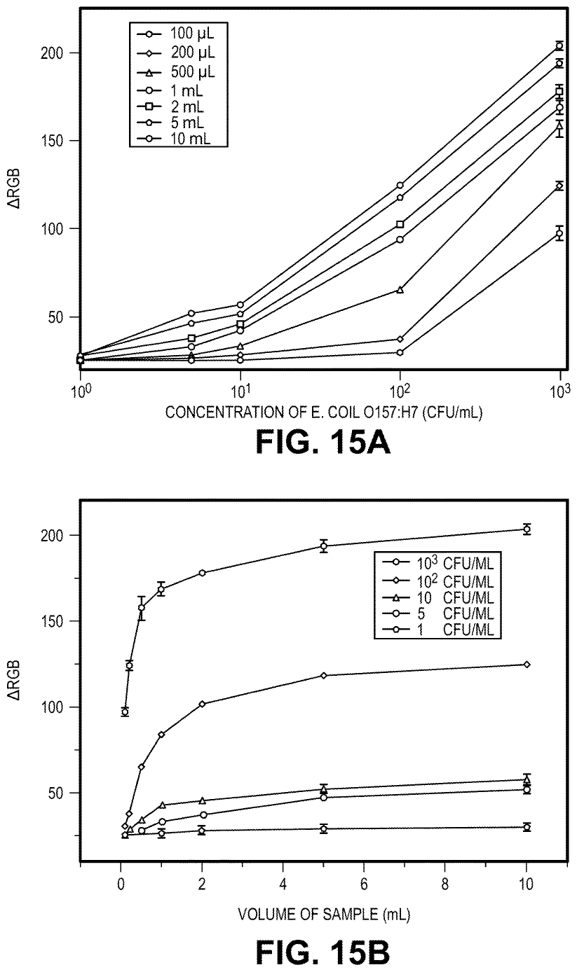

Traditional ELISA assays are typically operated within a limited range of sample volumes. Recent innovations, particularly with p-ELISA, have pivoted towards miniaturization, leading to even smaller sample volumes, which are suitable for most biological samples. However, unlike point-of-care clinical analysis, pathogens might exist at very low concentrations in various ground and surface water, treated industrial wastes, and food samples, large volumes of those samples are available and meaningful for detection of low concentrations of pathogens. Due to the fact that the MF membranes in varied thicknesses did not result in a significant increase in resistance to fluids (FIG. 11) and no none-specific retention of the targeted microorganism, amplifying the test sample volume allows for a larger number of pathogen binding on the MF structure, thereby increasing the signal intensities. Consequently, even trace amounts of pathogens become detectable when large volumes of test solutions are passed through the foam sensing material. The volume-responsive performances of the f-ELISA were extensively investigated with E. coli O157:H7 solution at different volumes: 100 μL, 200 μL, 500 μL, 1 mL, 2 mL, 5 mL, and 10 mL. Apart from the alterations in analyte volumes, all other testing steps were the same as the protocols used in the earlier discussions. As shown in FIG. 15a, it is clear that different volume sizes produced varying calibration curves. Importantly, when the sample volume was increased from 100 μL to 2 mL, the sensitivity of the f-ELISA in detecting E. coli O157:H7 improved significantly, reducing the LOD to 5 CFU/mL (FIG. 15b). Such a unique feature of the f-ELISA allows sensors to handle varied sample volumes and potentially serve as a flowing-through filtering sensor system for large-volume target solutions. Compared to other sensing materials listed in Table 1, the f-ELISA is capable of detecting trace amounts of E. coli O157:H7 in 56 min, making it more sensitive and less time-consuming than most other optical biosensors.

### 3.5 Enhanced Sensitivity Through Pre-Enrichment

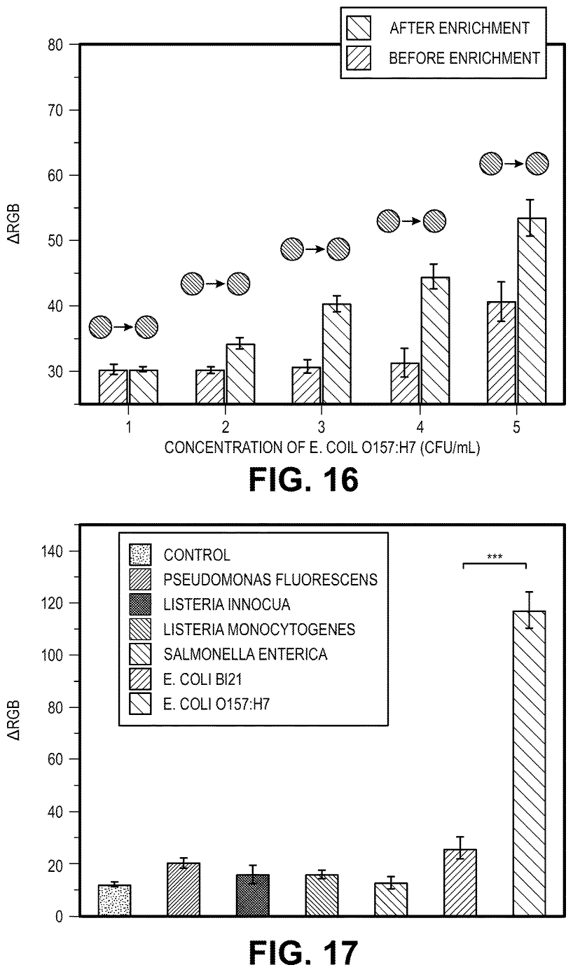

To further enhance the biosensor's sensitivity, we incubated the target specimen in the TSB medium for an hour under 37° C. prior to the f-ELISA detection. Consequently, the colorimetric signal of E. coli O157:H7 at a concentration of 2 CFU/mL became discernible through analysis of images taken by a smartphone (FIG. 16). Even though an extra hour is needed, the overall time taken by the f-ELISA-based biosensor, including incubation, remains under 2 hours, a duration that is relatively rapid for detecting bacteria concentrations.

TABLE 1
Comparison of optical biosensors for detection of E. coli O157:H7

| | | LOD | | | | |
|---|---|---|---|---|---|---|
| | Capture | (CFU/ | | | | |
| Sensor platform | reagent | mL) | Time | | Application | Ref |
| Whatman paper | Antibody | 10⁵ | <5 | hours | urine | 54 |
| Whatman filter paper | Antibody | 10⁴ | <3 | hours | lixivium samples | 55 |
| Wax-printed paper | Antibody | 10⁴ | 2.5 | hours | Beef samples | 56 |
| PDA vesicle | Antibody | 10⁴ | 2 | hours | Fecal samples | 57 |
| | | | | | and water | |
| 96 well plate | Antibody | 10⁴ | 2 | hours | Green tea sample | 58 |
| GO-Fe₃O₄ | Aptamer | 467 | 30 | min | Complex | 59 |
| | | | | | biological | |
| | | | | | samples | |
| Iron quantum cluster | amino | 8.3 × 10³ | 30 | min | Urine, tap water | 60 |
| | acids | | | | | |
| T-bacteriophage | PP0 1 ccp | 1 | 15 | hours | Apple juice | 61 |
| | phage | | | | | |
| Functionalized Gold | Reduce | 10² | 1 | hour | complex | 62 |
| NPs | exogenous | | | | artificial sepsis | |
| | | | | | blood | |
| Chemically modified | Antibody | 5 | 56 | min | Agricultural | This |
| MF | | | | | water and milk | work |
| Chemically modified | Antibody | 2 | 2 | hours | Agricultural | This |
| MF | | | | | water and milk | work |

*LOD = Limit of detection
*PDA = polydiacetylene
*GO = Graphene oxide

### 3.6 Selectivity of f-ELISA

The selectivity of the developed f-ELISA biosensing platform was evaluated using various bacterial strains: Pseudomonas fluorescens, Listeria innocua, Listeria monocytogenes, Salmonella enterica, and E. coli BL21. In our tests, only E. coli O157:H7 produced a discernible colorimetric signal, as illustrated in FIG. 17. Intriguingly, even a different strain of E. coli (BL21) failed to yield a significant signal, underscoring the good selectivity of the immobilized antibody on this biosensor. This specific reaction with only E. coli O157:H7 and the non-reactivity with other bacterial strains provides confidence in the applications of the f-ELISA-based biosensor. It offers the potential for accurate pathogen detection in real-world scenarios without being affected by the presence of other bacterial strains.

### 3.7 Real Sample Analysis

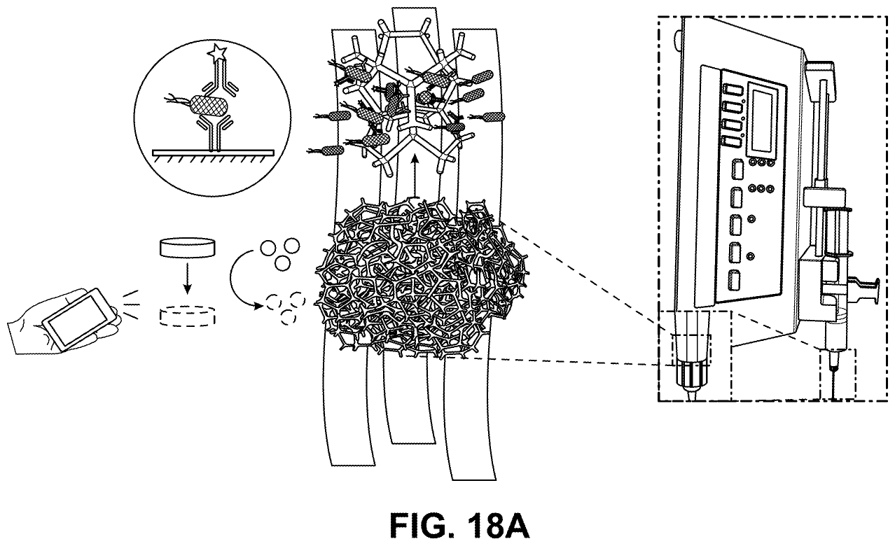

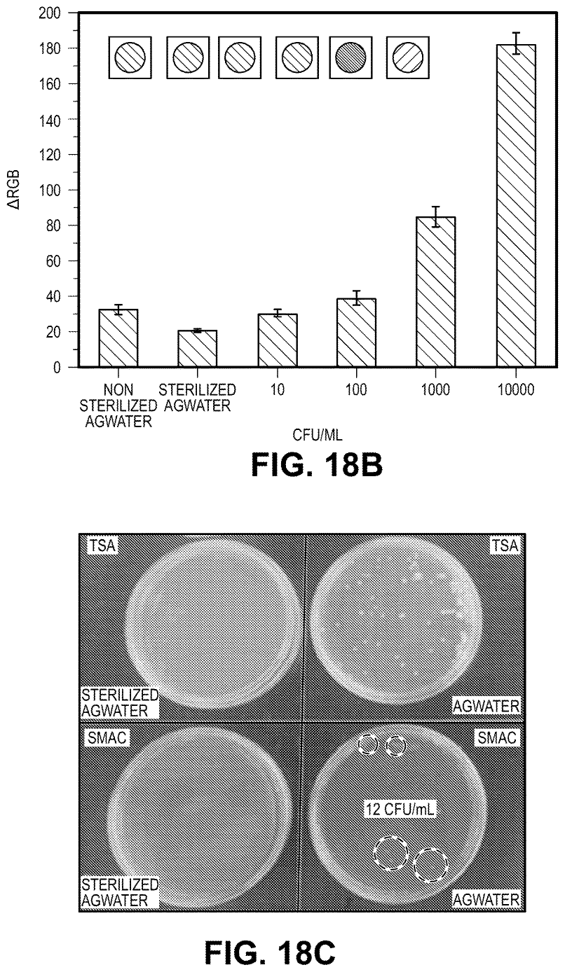

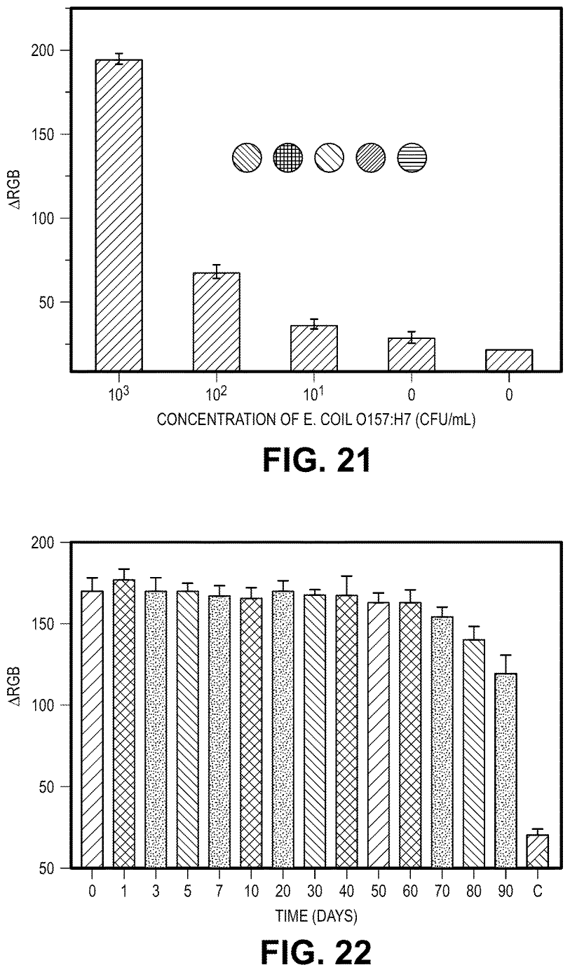

To investigate the efficiency of the f-ELISA in real-world scenarios of E. coli O157:H7 detection, we designed artificially contaminated milk samples bought from local grocery market and agricultural water (Agwater) collected from the irrigation facility at UC Davis, California. The MF membrane was mounted into a syringe needle pocket, 5 mL of sample solution was filled into a 20 mL syringe and flowed through the filtering needle with a flow rate of 10 mL/h, controlled by a SyringeONE programmable syringe pump (NewEra Instruments, USA) (FIG. 18a). For each test, E. coli O157:H7 was detectable at a concentration of 10 CFU/mL with a sample volume of 5 mL in a flowing-through filtering sensor system demonstrated in FIG. 8b and FIG. 21. Interestingly, for the non-sterilized agricultural water, the f-ELISA's colorimetric intensity was just above that of a 10 CFU/mL spiked sample. Subsequent culture plate assays confirmed the presence of E. coli O157:H7 at 12 CFU/mL in the agricultural water sample by using SMAC as a selective and differential medium for the detection of E. coli O157:H7, which aligns with the biosensor results (FIG. 8c). The bacteria colonies grown in the TSA medium indicated that there were some other strains of bacteria that existed in the Agwater (FIG. 17c). This instance effectively confirms the biosensing platform specificity, and precision. MF as a solid support is not affected by the matrices due to its unique structure features, facilitating the diffusion and flow of all large molecules existing in the mixture. Overall, the results suggest that the developed f-ELISA biosensor is reliable and accurate in detecting E. coli O157:H7 in complex matrices.

### 3.8 Storage Stability Evaluation for f-ELISA

Oxidation, isomerization, and hydrolysis prevent the antibody from existing long-term in the liquid state, diminishing the efficiency of the antibody-antigen interaction⁶³. In contrast, when antibodies are immobilized on a solid phase, they can retain their activity for an extended period⁶⁴. To ensure the long-term efficacy and repeatability of the f-ELISA system, we examined the stability and activity of the stored f-ELISA biosensing platform. The Ab@NHS@MF membranes were prepared using 10% sucrose as a stabilizer followed by freeze-drying⁶⁵. They were stored at a consistent temperature of 4° C. and assessed over a period of 90 days. At predetermined time intervals, sample membranes were retrieved and utilized in the f-ELISA assay to detect E. coli O157:H7 following the same protocols. The results indicate that the antibodies stored at 4° C. maintained their activity for up to 80 days, showing minimal variation from the results obtained with fresh Ab@NHS@MF membranes (FIG. 22). Therefore, with the employment of sucrose as a stabilizer for antibodies, prolonged storage without compromising the efficiency of the f-ELISA system can be achieved.

### 4. Conclusion

The development and evaluation of a novel f-ELISA biosensor for the detection of E. coli O157:H7 are presented here. This sensor, constructed with NHS@MF, possessing a unique reticulated three-dimensional (3D) macroporous framework structure, demonstrated high sensitivity, specificity, and selectivity, outperforming other conventional methods presented in literatures. The method required less than 56 min to complete the detection and demonstrated a sensitivity of 10 CFU/mL, with color signals discernible by the naked eye, and an enhanced sensitivity of 5 CFU/mL with the help of a smartphone. Following a brief bacteria enrichment period of 1 hour, the sensitivity was further amplified to 2 CFU/mL. Interestingly, the sensitivity increases as the volume of the sample increases, making this method highly suitable for testing large-volume samples, such as milk, agricultural water, etc. In essence, using E. coli O157:H7 as a proof of concept, this work not only paves the way for improved bacterial detection in environmental and food samples but also introduces f-ELISA as a new model that could be adapted for other pathogens and contaminants.

### REFERENCES

1. Reen, D. J. (1994). Enzyme-linked immunosorbent assay (ELISA). Basic Protein and Peptide Protocols, 461-466.
2. Shah, K., & Maghsoudlou, P. (2016). Enzyme-linked immunosorbent assay (ELISA): the basics. British journal of hospital medicine, 77(7), C98-C101.
3. Wu, L., Li, G., Xu, X., Zhu, L., Huang, R., & Chen, X. (2019). Application of nano-ELISA in food analysis: recent advances and challenges. TrAC Trends in Analytical Chemistry, 113, 140-156.
4. Cheng, C. M., Martinez, A. W., Gong, J., Mace, C. R., Phillips, S. T., Carrilho, E., . . . & Whitesides, G. M. (2010). Paper-based ELISA. Angewandte Chemie, 122(28), 4881-4884.
5. Zhao, C., Pan, B., Wang, M., Si, Y., Taha, A. Y., Liu, G., . . . & Sun, G. (2022). Improving the Sensitivity of Nanofibrous Membrane-Based ELISA for On-Site Antibiotics Detection. ACS sensors, 7(5), 1458-1466.
6. Zhao. C., Si, Y., Zhu. S., Bradley, K., Taha, A. Y., Pan, T., & Sun, G. (2021). Diffusion of protein molecules through microporous nanofibrous poly acrylonitrile membranes. ACS applied polymer materials, 3(3). 1618-1627.
7. Martinez, A. W. (2011). Microfluidic paper-based analytical devices: from POCKET to paper-based ELISA. Bioanalysis, 3(23), 2589-2592.
8. Jung, I. Y., Kim. J. S., Choi, B. R., Lee, K., & Lee, H. (2017). Hydrogel based biosensors for in vitro diagnostics of biochemicals, proteins, and genes. Advanced healthcare materials, 6(12), 1601475.
9. Power, M., Hosticka, B., Black, E., Daitch, C., & Norris, P. (2001). Aerogels as biosensors: viral particle detection by bacteria immobilized on large pore aerogel. Journal of Non-Crystalline Solids, 285(1-3), 303-308.
10. Fang, L. X., Huang, K. J., & Liu. Y. (2015). Novel electrochemical dual-aptamer-based sandwich biosensor using molybdenum disulfide/carbon aerogel composites and Au nanoparticles for signal amplification. Biosensors and Bioelectronics, 71, 171-178.
11. Gao, K., Guo, Y., Niu, Q., Fang, H., Zhang, L., Zhang, Y., . . . & Zhou, L. (2018). Effects of chitin nanofibers on the microstructure and properties of cellulose nanofibers/chitin nanofibers composite aerogels. Cellulose, 25(8). 4591-4602.
12. Feng, Y., & Yao, J. (2018). Design of melamine sponge-based three-dimensional porous materials toward applications. Industrial & Engineering Chemistry Research, 57(22), 7322-7330.
13. Oribayo, O., Feng, X., Rempel. G. L., & Pan, Q. (2017). Modification of formaldehyde-melamine-sodium bisulfite copolymer foam and its application as effective sorbents for clean up of oil spills. Chemical Engineering Science, 160, 384-395.
14. Stolz. A., Le Floch. S., Reinert, L., Ramos, S. M., Tuaillon-Combes, J., Soneda, Y., & San-Miguel, A. (2016). Melamine-derived carbon sponges for oil-water separation. Carbon, 107, 198-208.
15. Zhao, S. W., Zhou, Q., Long, N. B., & Zhang, R. F. (2022). Efficient synthesis of N-acetyllactosamine using immobilized β-galactosidase on a novel 3D polymer support. Enzyme and Microbial Technology, 110070.
16. Ji, B., Zhao, W., Xu, X., Han, Y., Jie, M., Xu, G., & Bai, Y. (2021). Development of a modified quick, easy, cheap, effective, rugged, and safe method based on melamine sponge for multi-residue analysis of veterinary drugs in milks by ultra-performance liquid chromatography tandem mass spectrometry. Journal of Chromatography A, 1651, 462333.
17. Zhao, C., Si, Y., Pan, B., Taha, A. Y., Pan, T., & Sun, G. (2020). Design and fabrication of a highly sensitive and naked-eye distinguishable colorimetric biosensor for chloramphenicol detection by using ELISA on nanofibrous membranes. Talanta, 217, 121054.
18. Nguyen, Y., & Sperandio, V. (2012). Enterohemorrhagic E. coli (EHEC) pathogenesis. Frontiers in cellular and infection microbiology, 2, 90.
19. Riley. L. W., Remis, R. S., Helgerson, S. D., McGee, H. B., Wells, J. G., Davis, B. R., . . . & Cohen, M. L. (1983). Hemorrhagic colitis associated with a rare Escherichia coli serotype. New England journal of medicine. 308(12), 681-685.
20. Griffin, P. M., Ostroff, S. M., Tauxe, R. V., Greene, K. D., Wells, J. G., Lewis, J. H., & Blake, P. A. (1988). Illnesses associated with Escherichia coli O157: H7 infections: a broad clinical spectrum. Annals of internal medicine, 109(9), 705-712.
21. Tapia, D., Ross, B. N., Kalita, A., Kalita. M., Hatcher, C. L., Muruato, L. A., & Torres, A. G. (2016). From in silico protein epitope density prediction to testing Escherichia coli O157: H7 vaccine candidates in a murine model of colonization. Frontiers in Cellular and Infection Microbiology, 6, 94.
22. Ameer M A, Wasey A, Salen P. Escherichia coli (E. coli O157 H7) [Updated 2023 Aug. 8]. In: StatPearls [Internet]. Treasure Island (FL): StatPearls Publishing; 2023 January-. Available from: https://www.ncbi.nlm.nih.gov/books/NBK507845/
23. Doyle, M. P. (1991). Escherichia coli O157: H7 and its significance in foods. International journal of food microbiology, 12(4), 289-301.
24. Bavaro, M. F. (2012). E. coli O157: H7 and other toxigenic strains: the curse of global food distribution. Current gastroenterology reports, 14, 317-323.
25. Saxena, T., Kaushik, P., & Mohan, M. K. (2015). Prevalence of E. coli O157: H7 in water sources: an overview on associated diseases, outbreaks and detection methods. Diagnostic microbiology and infectious disease, 82(3), 249-264.
26. Deisingh, A. K., & Thompson, M. (2004). Strategies for the detection of Escherichia coli O157: H7 in foods. Journal of applied microbiology, 96(3), 419-429.
27. Notomi. T., Okayama, H., Masubuchi, H., Yonekawa, T., Watanabe, K., Amino, N., & Hase, T. (2000). Loop-mediated isothermal amplification of DNA. Nucleic acids research, 28(12), e63-e63.
28. Tomita. N., Mori. Y., Kanda. H., & Notomi, T. (2008). Loop-mediated isothermal amplification (LAMP) of gene sequences and simple visual detection of products. Nature protocols. 3(5). 877-882.
29. Zadik, P. M., Chapman, P. A., & Siddons, C. A. (1993). Use of tellurite for the selection of verocytotoxigenic Escherichia coli O157. Journal of Medical Microbiology, 39(2), 155-158.
30. Sanderson, M. W., Gay, J. M., Hancock, D. D., Gay. C. C., Fox, L. K., & Besser, T. E. (1995). Sensitivity of bacteriologic culture for detection of Escherichia coli O157: H7 in bovine feces. Journal of Clinical Microbiology, 33(10), 2616-2619.
31. Bayardelle, P., & Zafarullah, M. (2002). Development of oligonucleotide primers for the specific PCR-based detection of the most frequent Enterobacteriaceae species DNA using wec gene templates. Canadian journal of microbiology, 48(2), 113-122.
32. Campbell, G. R., Prosser, J., Glover, A., & Killham, K. (2001). Detection of Escherichia coli O157: H7 in soil and water using multiplex PCR. Journal of Applied Microbiology, 91(6), 1004-1010.
33. Li, Y., Fan, P., Zhou, S., & Zhang, L. (2017). Loop-mediated isothermal amplification (LAMP): A novel rapid detection platform for pathogens. Microbial pathogenesis, 107, 54-61.
34. Karo, O., Wahl, A., Nicol, S. B., Brachert, J., Lambrecht, B., Spengler, H. P., . . . & Montag, T. (2008). Bacteria detection by flow cytometry. Clinical chemistry and laboratory medicine. 46(7), 947-953.
35. Davis, R., Irudavaraj, J., Reuhs, B. L., & Mauer, L. J. (2010). Detection of E. coli O157: H7 from ground beef using fourier transform infrared (FT-IR) spectroscopy and chemometrics. Journal of Food Science, 75(6), M340-M346.
36. Gous, N., Boeras, D. I., Cheng, B., Takle, J., Cunningham, B., & Peeling, R. W. (2018). The impact of digital technologies on point-of-care diagnostics in resource-limited settings. Expert review of molecular diagnostics, 18(4), 385-397.
37. Rubach. M. P., Halliday, J. E., Cleaveland. S., & Crump, J. A. (2013). Brucellosis in low-income and middle-income countries. Current opinion in infectious diseases, 26(5), 404.
38. Nguyen. Q. H., & Kim, M. I. (2020). Nanomaterial-mediated paper-based biosensors for colorimetric pathogen detection. TrAC Trends in Analytical Chemistry, 132, 116038.
39. Hu, J., Wang, S., Wang, L., Li, F., Pingguan-Murphy, B., Lu, T. J., & Xu, F. (2014). Advances in paper-based point-of-care diagnostics. Biosensors and Bioelectronics, 54, 585-597.
40. Zhao, C., Si, Y., Pan, B., Taha, A. Y., Pan, T., & Sun, G. (2020). Design and fabrication of a highly sensitive and naked-eye distinguishable colorimetric biosensor for chloramphenicol detection by using ELISA on nanofibrous membranes. Talanta, 217, 121054.
41. Yu, X., Pan, B., Zhao, C., Shorty, D., Solano, L. N., Sun, G., . . . & Lam, K. S. (2023). Discovery of Peptidic Ligands against the SARS-CoV-2 Spike Protein and Their Use in the Development of a Highly Sensitive Personal Use Colorimetric COVID-19 Biosensor. ACS sensors.
42. Zhao, C., Pan, B., Wang, M., Si, Y., Taha, A. Y., Liu, G., . . . & Sun, G. (2022). Improving the sensitivity of nanofibrous membrane-based ELISA for on-site antibiotics detection. ACS sensors, 7(5), 1458-1466.
43. Zhao, C., Si, Y., Zhu. S., Bradley, K., Taha, A. Y., Pan, T., & Sun, G. (2021). Diffusion of protein molecules through microporous nanofibrous polyacrylonitrile membranes. ACS applied polymer materials, 3(3), 1618-1627.
44. Tang, R., Xie, M. Y., Li, M., Cao, L., Feng, S., Li, Z., & Xu, F. (2022). Nitrocellulose membrane for paper-based biosensor. Applied Materials Today, 26, 101305.
45. Pan. B., Zhao, C., Norwood, M., Wang, M., Y. Liu, G., & Sun, G. (2023). Highly Sensitive Naked Eye Detectable Colorimetric Biosensors Made from Macroporous Framework Melamine Foams for Onsite and Simultaneous Detection of Multiple Environmental Hazards in Flowing Through Sensing Systems. Advanced Sensor Research, 2300080.
46. Koch, A. L. (1970). Turbidity measurements of bacterial cultures in some available commercial instruments. Analytical biochemistry, 38(1), 252-259.
47. El-Moghazv, A. Y., Wisuthiphaet, N., Amaly, N., & Nitin, N. (2022). Enhanced sampling of bacteria and their biofilms from food contact surfaces with robust cationic modified swabs. Cellulose. 29(8), 4509-4524.
48. Cleland, J. L., Lam, X., Kendrick, B., Yang, J., Yang, T. H., Overcashier, D., . . . & Carpenter, J. F. (2001). A specific molar ratio of stabilizer to protein is required for storage stability of a lyophilized monoclonal antibody. Journal of pharmaceutical sciences, 90(3), 310-321.
49. Chen, C. A., Yeh, W. S., Tsai, T. T., & Chen, C. F. (2019). Three-dimensional origami paper-based device for portable immunoassay applications. Lab on a Chip, 19(4), 598-607.
50. Schneider, C. A., Rasband, W. S., & Eliceiri, K. W. (2012). NIH Image to ImageJ: 25 years of image analysis. Nature methods. 9(7), 671-675.
51. Murdock, R. C., Shen, L., Griffin, D. K., Kelley-Loughnane, N., Papautsky, I., & Hagen, J. A. (2013). Optimization of a paper-based ELISA for a human performance biomarker. Analytical chemistry, 85(23), 11634-11642.
52. Levin, P. A., & Angert, E. R. (2015). Small but mighty: cell size and bacteria. Cold Spring Harbor perspectives in biology, 7(7), a019216.
53. Rehman, I., & Bonfield, W. J. J. (1997). Characterization of hydroxyapatite and carbonated apatite by photo acoustic FTIR spectroscopy. Journal of materials science: Materials in medicine, 8(1), 1-4.
54. Shih, C. M., Chang, C. L., Hsu, M. Y., Lin, J. Y., Kuan, C. M., Wang, H. K., . . . & Cheng, C. M. (2015). Paper-based ELISA to rapidly detect Escherichia coli. Talanta, 145, 2-5.
55. Pang, B., Zhao, C., Li, L., Song, X., Xu, K., Wang, J., . . . & Li, J. (2018). Development of a low-cost paper-based ELISA method for rapid Escherichia coli O157: H7 detection. Analytical Biochemistry, 542, 58-62.
56. Zhao. Y., Zeng. D., Yan, C., Chen. W., Ren. J., Jiang, Y., . . . & Dai, J. (2020). Rapid and accurate detection of Escherichia coli O157: H7 in beef using microfluidic wax-printed paper-based ELISA. Analyst, 145(8), 3106-3115.
57. Wu, W., Zhang, J., Zheng, M., Zhong, Y., Yang, J., Zhao, Y., . . . & Lu, J. (2012). An aptamer-based biosensor for colorimetric detection of Escherichia coli O157: H7. PloS one, 7(11), e48999.
58. Feng, M., Yong, Q., Wang, W., Kuang, H., Wang, L., & Xu, C. (2013). Development of a monoclonal antibody-based ELISA to detect Escherichia coli O157: H7. Food and agricultural immunology, 24(4), 481-487.
59. Yao, Y., Xie, G., Zhang, X., Yuan. J., Hou, Y., & Chen, H. (2021). Fast detection of E. coli with a novel fluorescent biosensor based on a FRET system between UCNPs and GO@Fe 3 O 4 in urine specimens. Analytical Methods. 13(19). 2209-2214.
60. Vaezi, Z., Azizi, M., Mohammadi, S. S., Hashemi, N., & Naderi-Manesh, H. (2020). A novel iron quantum cluster confined in hemoglobin as fluorescent sensor for rapid detection of Escherichia coli. Talanta, 218, 121137.
61. Hoang, H. A., & Dien, L. T. (2015). Rapid and simple colorimetric detection of Escherichia coli O157: H7 in apple juice using a novel recombinant bacteriophage-based method. Biocontrol science, 20(2), 99-103.
62. Mou, X. Z., Chen, X. Y., Wang, J., Zhang. Z., Yang, Y., Shou, Z. X., . . . & Li. Y. Q. (2019). Bacteria-instructed click chemistry between functionalized gold nanoparticles for point-of-care microbial detection. ACS applied materials & interfaces, 11(26), 23093-23101.
63. Allison, S. D., Manning, M. C., Randolph, T. W., Middleton, K., Davis, A., & Carpenter, J. F. (2000). Optimization of storage stability of lyophilized actin using combinations of disaccharides and dextran. Journal of pharmaceutical sciences, 89(2), 199-214.
64. Wang, J., Yiu, B., Obermeyer, J., Filipe, C. D., Brennan, J. D., & Pelton, R. (2012). Effects of temperature and relative humidity on the stability of paper-immobilized antibodies. Biomacromolecules, 13(2). 559-564.
65. Chen, C. A., Yeh, W. S., Tsai, T. T., & Chen, C. F. (2019). Three-dimensional origami paper-based device for portable immunoassay applications. Lab on a Chip, 19(4), 598-607.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A three-dimensional (3-D) macroporous melamine foam membrane comprising a secondary amine reactive group.

2. The membrane of claim 1, wherein the secondary amine reactive group is a member selected from the group consisting of an activated ester, a maleimide and a pyridyldithiol.

3. The membrane of claim 2, wherein the activated ester is installed using a cross-linking agent selected from the group consisting of DSC (N,N′-disuccinimidyl carbonate), DSG (disuccinimidyl glutarate, DSS (disuccinimidyl suberate), BS3 (bis(sulfosuccinimidyl)suberate), BS(PEG)9 (PEGylated bis(sulfosuccinimidyl)suberate), DSP (dithiobis(succinimidyl propionate)), DTSSP (3,3′-dithiobis(sulfosuccinimidyl propionate)), EGS (ethylene glycol bis(succinimidyl succinate)) and a combination thereof.

4. The membrane of claim 3, wherein the activated ester is installed using N, N′-disuccinimidyl carbonate (DSC) as the cross-linking agent.

5. The membrane of claim 1, wherein the microporous melamine foam has pore sizes of about 60 μm to about 150 μm.

6. The membrane of claim 1, wherein the melamine foam is modified with a biomolecule using the amine reactive coupling reagent.

7. The membrane of claim 6, wherein the biomolecule is a member selected from the group consisting of a protein, a peptide, a hormone, an antibody, an antigen, a hapten, a carbohydrate, and a ligand.

8. The membrane of claim 7, wherein the biomolecule is an epitope of the antigen.

9. A sandwich ELISA method for determining the presence of an analyte in solution, the method comprising:
contacting a macroporous melamine foam membrane comprising an immobilized capture antibody with a sample containing an analyte, wherein the capture antibody is specific for an epitope of the analyte to form a captured analyte;
contacting the captured analyte with a detection antibody to form a sandwiched moiety; and
detecting an output signal from the sandwiched moiety.

10. The sandwich ELISA method of claim 9, wherein the detection antibody is labeled.

11. The sandwich ELISA method of claim 9, wherein the detection antibody is unlabeled.

12. The sandwich ELISA method of claim 11, further comprising a secondary enzyme-conjugated detection antibody.

13. The sandwich ELISA method of claim 9, wherein the analyte is a foodborne pathogen.

14. A direct ELISA method for determining the presence of an analyte in solution, the method comprising:
contacting a macroporous melamine foam membrane comprising an immobilized analyte with a capture antibody, wherein the capture antibody is specific for an epitope of the analyte to form a captured analyte; and
detecting an output signal from the captured analyte.

15. The direct ELISA method of claim 14, wherein the capture antibody comprises a label.

16. The direct ELISA method of claim 15, wherein the label comprises an enzyme.

17. The direct ELISA method of claim 14, wherein the analyte is a foodborne pathogen.

18. A competitive ELISA method for determining the amount an analyte in sample, the method comprising:
contacting a macroporous melamine foam membrane comprising an immobilized capture antibody with a sample containing the analyte and a conjugated analyte, wherein the analyte competes with the conjugated analyte in the sample for the immobilized capture antibody to form a captured analyte; and
detecting an output signal from the captured analyte.

19. The competitive ELISA method of claim 18, wherein the capture antibody optionally comprises a label.

20. The competitive ELISA method of claim 18, wherein the conjugated antigen optionally comprises a label.

21. The competitive ELISA method of claim 18, wherein the capture antibody comprises a label.

22. The competitive ELISA method of claim 18, wherein the conjugated antigen comprises a label.

23. The competitive ELISA method of claim 21 or 22, wherein the label comprises an enzyme.

24. The competitive ELISA method of claim 18, wherein the higher the sample antigen concentration, the weaker the output signal, indicating that the signal output inversely correlates with the amount of antigen in the sample.

25. The competitive ELISA method of claim 18, wherein the analyte is a foodborne pathogen.

26. A sandwich ELISA method for determining the presence of a foodborne pathogen in solution, the method comprising:
contacting a macroporous melamine foam membrane comprising an immobilized capture antibody with a sample containing a foodborne pathogen, wherein the capture antibody is specific for an epitope of the analyte to form a captured foodborne pathogen;
contacting the captured foodborne pathogen with a detection antibody to form a sandwiched moiety; and
detecting an output signal from the sandwiched moiety.

27. The sandwich ELISA method of claim 26, wherein the pathogen is a member selected from the group consisting of Staphylococcus aureus, Salmonella, Clostridium perfringens, Campylobacter, Listeria monocytogenes, Vibrio parahaemolyticus, Bacillus cereus, and Entero-pathogenic Escherichia coli.

28. A direct ELISA method for determining the presence of a foodborne pathogen in solution, the method comprising:
contacting a macroporous melamine foam membrane comprising an immobilized foodborne pathogen with a capture antibody, wherein the capture antibody is specific for an epitope of the foodborne pathogen to form a captured foodborne pathogen; and
detecting an output signal from the captured foodborne pathogen.

29. The direct ELISA method of claim 28, wherein the pathogen is a member selected from the group consisting of Staphylococcus aureus, Salmonella, Clostridium perfringens, Campylobacter, Listeria monocytogenes, Vibrio parahaemolyticus, Bacillus cereus, and Entero-pathogenic Escherichia coli.

30. A competitive ELISA method for determining the amount a foodborne pathogen in a sample, the method comprising:
contacting a macroporous melamine foam membrane comprising an immobilized capture antibody with a sample containing the foodborne pathogen and a conjugated foodborne pathogen, wherein the conjugated foodborne pathogen competes with the foodborne pathogen in the sample for the capture antibody to form a captured analyte; and
detecting an output signal from the captured foodborne pathogen.

31. The competitive ELISA method of claim 30, wherein the capture antibody optionally comprises a label.

32. The competitive ELISA method of claim 30, wherein the conjugated foodborne pathogen optionally comprises a label.

33. The competitive ELISA method of claim 30, wherein the capture antibody comprises a label.

34. The competitive ELISA method of claim 30, wherein the conjugated foodborne pathogen comprises a label.

35. The competitive ELISA method of claim 33 or 34, wherein the label comprises an enzyme.

36. The competitive ELISA method of claim 28, wherein the pathogen is a member selected from the group consisting of Staphylococcus aureus, Salmonella, Clostridium perfringens, Campylobacter, Listeria monocytogenes, Vibrio parahaemolyticus, Bacillus cereus, and Entero-pathogenic Escherichia coli.
